(19) United States
(12) Patent Application Publication
(10) Pub. No.: US 2011/0028427 A1
(43) Pub. Date: Feb. 3, 2011

(54) DIETARY FIBER AND METHOD FOR PREPARING DIETARY FIBER

(75) Inventor: David Ellis McManus, Melbourne (AU)

Correspondence Address: NIXON PEABODY LLP - PATENT GROUP, 1100 CLINTON SQUARE, ROCHESTER, NY, 14604, US

(73) Assignee: NUTRIFINA PTY LTD, Melbourne, Victoria (AU)

(21) Appl. No.: 12/746,106

(22) PCT Filed: Dec. 4, 2008
(86) PCT No.: PCT/AU2008/001790

§ 371 Date: Sep. 14, 2010

Related U.S. Application Data
(60) Provisional application No. 60/996,786, filed on Dec. 5, 2007.

(51) Int. Cl. A61K 31/715; A23L 1/212; A23L 1/308

(52) U.S. Cl. 514/54; 426/238; 426/425; 426/243; 426/270; 426/321; 426/542; 426/98

(57) ABSTRACT

The invention relates to a soluble antioxidant dietary fiber obtained from pineapple and a method of processing pineapple pulp to provide a soluble antioxidant fiber.

### FIELD

This invention relates to a dietary fiber and a method for preparing dietary fiber. In particular this invention relates to preparation of a soluble dietary fiber having high antioxidant activity.

### BACKGROUND

It is well accepted in the medical community that fruit, vegetables and whole grain cereals are important to a healthy diet due to the relatively high associated intake of dietary fiber. Dietary fiber has established benefits for reducing cholesterol, lowering risk of cardiovascular disease, stroke, type 2 diabetes and many other chronic illnesses. One of the benefits of a diet rich in fruit, vegetables and whole grains is also partly attributed to the relatively high associated intake of dietary polyphenols especially hydroxycinnamates and flavonoids. Consumption of polyphenols is beneficial for reduced risk of cardio-vascular disease, cerebrovascular disease, type 2 diabetes, cancers of the lung and prostate, and other chronic illnesses. The antioxidant and anti-inflammatory properties of dietary polyphenols are also believed to have benefits for other chronic diseases involving oxidative stress or inflammation.

Attempts have been made to replicate the health and dietary benefits of vegetables, wholegrains and fruit by using supplements containing purified phytochemicals. However recently a number of authors including Liu [J. Nutr. 134 (2004) 3479S-3485S] have shown that the interactions cannot be adequately replicated. While not fully understood, the association between dietary fiber and prevention of disease is thought to be due to synergistic interactions between dietary fiber antioxidants and other biologically active components.

The US Heart Association recommends a total dietary fiber intake of 25-30 grams per day from foods and not supplements. Currently the dietary fiber intake of adults in the US is about half the recommended amount. This difference, commonly referred to as “the fiber gap”, is a serious problem for public health.

Consumers tend to rely on fruit and vegetable juices as a significant component of daily consumption. While these products are seen as being healthy and more convenient than whole fruit and vegetables, the American Heart Association has warned that the nutritional value of fruit and vegetables is lost during juice processing and recommends that health professionals should not emphasise juice consumption. During preparation of apple juice for example, most of the fiber and polyphenols are retained in the pomace rather than being transferred to the juice. In order to consume the fiber and polyphenols associated with one whole apple it is therefore necessary to drink three 250 ml servings of apple juice.

Dietary fiber has been proposed for addition to fruit drinks as a supplement to the loss of dietary fiber. Some popular dietary fibers such as inulin suffer from the disadvantage of being unstable under conditions of low pH and high temperature. They are therefore hydrolysed during pasteurisation. Klewicki has reported in Food Science Technology 40 (2007) 1259-1265 that the use of inulin in preparing fruit and vegetables drinks results in a lower than expected fiber level and higher than expected sugar levels.

Attempts have also been made to supplement dietary polyphenols. However, plant based phenols, flavonoids, iso-flavonoids, turpenes and glucose inolates are almost always bitter, acrid or astringent. Processed additives therefore are unattractive for the consumer, posing a dilemma for the food industry.

Attempts have been made to isolate antioxidant dietary fiber from fruits and vegetables. However, the problem encountered is that the processed fruit fiber products currently available are not truly soluble. All the current products are presently in the form of washed “fruit flours” containing 30% to 50% of insoluble fiber. This tends to result in a thick grainy coarse beverage which is less palatable and acceptable to customers.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY

We have now found that by subjecting pineapple pulp to extraction by heating of a pulp of acid pH it is possible to extract a soluble dietary fiber containing phenolic antioxidants.

Accordingly, we provide a method of preparing a water soluble antioxidant dietary fiber comprising providing pineapple and heating the pulp to a temperature in the range of from 105° C. to 150° C. more preferably from 110° C. to 145° C. and most preferably 120° C. to 140° C.; and removing the soluble fiber component.

Preferably the pulp will be heated for a period of at least 30 seconds, preferably from 3 minutes to 5 hours and more preferably from 5 minutes to 2 hours. In the preferred embodiment the pulp, following heating, is subject to mechanical or ultrasonic treatment and separation of the soluble fiber from the solids.

The heating will in one embodiment involve maintaining a temperature in the range of from 90° C. to 150° C. for a period of at least 10 minutes and preferably from 10 minutes to 5 hours including said temperature in the range of from 105 to 150° C. more preferably from 110° C. to 145° C. and most preferably 120° C. to 140° C. for a period of at least 30 seconds, preferably from 3 minutes to 5 hours and more preferably from 5 minutes to 2 hours.

The process may further comprise the steps of removing free sugars and concentrating the soluble antioxidant fiber.

In a particularly preferred embodiment the invention provides a method of preparing an antioxidant dietary fiber comprising:
heating pineapple pulp to a temperature in the range from 105° C. to 150° C., preferably from 110° C. to 145° C. and most preferably from 120° C. to 140° C. for a period of at least 30 seconds; preferably from 3 minutes to 5 hours and more preferably from 5 minutes to 2 hours;
providing a pH of the autoclaved pineapple pulp in the range of from 3.2 to 6.5; preferably from 3.5 to 5.6 and more preferably from 3.5 to 4.5;
subjecting the heated material to mechanical or ultrasonic treatment to facilitate removal of soluble material from the solid material;
separation of the solubilised fiber from insoluble material;
at least partly removing free sugars; and
concentrating the soluble antioxidant fiber.

In a further aspect the invention provides a soluble antioxidant fiber obtained from pineapple. The soluble antioxidant dietary fiber is preferably characterised by having been obtained by autoclaving a pineapple pulp to achieve a pH in the range of from 3.2 to 6.5, preferably from 3.5 to 5.6 and most preferably from 3.5 to 4.5 at a temperature in the range of from 105° C. to 150° C. and more preferably from 110° C. to 145° C. The material will be autoclaved for a time sufficient to at least partly solubilise the antioxidant dietary fiber which would preferably be at least 30 seconds and more preferably from in the range of from 3 minutes to 5 hours.

The heating will in one embodiment involve maintaining a temperature in the range of from 90° C. to 150° C. for a period of at least 10 minutes and preferably from 10 minutes to 5 hours including said temperature in the range of from 105° C. to 150° C. more preferably from 110° C. to 145° C. and most preferably 120° C. to 140° C. for a period of at least 30 seconds, preferably from 3 minutes to 5 hours and more preferably from 5 minutes to 2 hours. While the temperature of at least 105° C. to 150° C. more preferably from 110° C. to 145° C. and most preferably 120° C. to 140° C. for at least 30 seconds (preferably at least 3 minutes and more preferably at least 5 minutes) is important to facilitate release of the antioxidant dietary fiber from the pulp the maintenance of the temperature in the range of from 90° C. to 150° C. for an additional period is useful to optimise the separation and/or to allow the solubilised material to disperse from the insoluble material.

Throughout the description and the claims of this specification the word “comprise” and variations of the word, such as “comprising” and “comprises” is not intended to exclude other additives, components, integers or steps.

### DETAILED DESCRIPTION

The present invention involves soluble dietary fiber obtained from pineapple and a method of obtaining soluble dietary fiber from pineapple. We have found that pineapple is unique among fruit and vegetables in allowing a higher level of antioxidant activity to be obtained in soluble dietary fiber.

Previously, soluble dietary fiber and associated phenolic compounds were extracted from a range of fruits, vegetables and cereal bran by Hartley [Am. J. Clin. Nutr. 31 (1978) S90-S93]. Extraction was carried out in 1N sodium hydroxide (4% w/v) under nitrogen, at 20° C. for 20 hours. Hartley reported that pineapple cell walls contained the highest level of phenolic compounds of all the materials tested. Pineapple has been reported to be very rich in hemicelluloses [Vidal-Valverde et al J. Food Sci. 47 (1982) 1840-1845]. The hemicellulose of pineapple is known to contain ester-linked ferulic acid [Smith and Harris, Plant Physiol. 107 (1995) 1399-1409], but the antioxidant content of pineapple is generally ranked low compared to other fruits and vegetables; for example Sun et al [J. Agric. Food Chem. 50 (2002) 7449-7454], Guo et al [Nutr. Res. 23 (2003) 1719-1726], Wu et al [J. Agric. Food Chem. 52 (2004) 4046-4037] and Garcia-Alonso et al [Food. Chem. 84 (2004) 13-18].

Historically, the term “hemicellulose” was applied to non-cellulosic wall polysaccharides other than pectins, which can be extracted in alkaline solutions, typically 1-4 M [Huisman et al Carbohydr. Polym. 42 (2000) 185-191]. The hemicelluloses are now known to comprise a diverse group of polysaccharides, including xyloglucans, gluco- and galactoglucomannans, galactomannans, (1→3)-beta-glucans and glucuronoarabinoxylans.

The glucuronoarabinoxylans (usually simply called “arabinoxylans”) often also contain phenolic acids, such as ferulic acids and p-coumaric acids. These are covalently bound through ester linkages to arabinose sidechains along the main polysaccharide backbone [Saulnier & Thibault J. Sci. Food Agric. 79 (1999) 396-402]. For example, wheat arabinoxylan is rich in ferulic acid, oat and maize arabinoxylans contain substantial proportions of both ferulic acid and p-coumaric acid, while psyllium arabinoxylan contains no detectable phenolic acids [Gioacchini et al J. Chromatogr. A 730 (1996) 31-37].

Phenolic acids present in the cell wall are thought to play an important role in the connection of polysaccharides with other cell wall components, including lignin, proteins and other polysaccharides. Such coupling reactions, probably catalysed by wall-bound peroxidises, create a cross-linked matrix structure which may be poorly digestible. The phenolic acids bound to cereal arabinoxylans account for at least some of the antioxidant activity that is associated with cereal brans [Liyana-Pathirana and Shahidi J. Agric. Food Chem. 54 (2006) 1256-1264].

Antioxidant-rich soluble dietary fiber can be recovered from cereal grains by hot water extraction, but the yield is very low (Bunzel et al J. Sci. Food Agric. 81 (2001) 653-660]. Treatment with hemicellulase enzymes produces feruloylated oligosaccharides, but not high molecular weight dietary fiber [Yuan et al Food Chem. 90 (2005) 759-764]. Extraction under mild alkaline conditions can release feruloylated arabinoxylans, but at low yield. It is generally known that, under alkaline conditions, ferulic acid is stripped off the hemicellulose backbone faster than the hemicellulose can be extracted [Mandalari et al J. Cereal Sci. 42 (2005) 205-212]. Extraction of hemicellulose under mild acidic conditions is known to result in cleavage of feruloylated arabinose sidechains from the hemicellulose backbone, causing loss of antioxidant activity. The antioxidant-rich sidechains can be liberated by autolysis through boiling of naturally acidic hemicellulose [Whistler & McGilvray J. Am. Chem. Soc. 77 (1955) 1884-1885], by mild hydrolysis with dilute sulphuric acid at room temperature [Whistler & Corbett J. Am. Chem. Soc. 77 (1955) 6328-6330], or by boiling at pH 2 [Wallace et al Carbohydr. Res. 272 (1995) 41-53].

We have found that a soluble dietary fiber can be extracted from pineapple pulp, such fiber having an antioxidant activity equivalent to 150-650 mg Vitamin E per gram due to the presence of covalently bound ferulic acid and p-coumaric acid. This is surprising, given that a high content of ferulic acid groups is supposedly responsible for the tightly-knit, dense structure of wheat bran while, in contrast, pineapple pulp has a soft, open structure and is reported to have a low antioxidant content.

We have also found that production of pineapple dietary fiber can best be accomplished under mild acidic conditions at high temperature. This is also surprising, since the prior art suggests that feruloylated arabinose sidechains will be stripped off the hemicellulose backbone under such conditions.

The present invention involves the preparation of soluble antioxidant dietary fiber by subjecting pineapple pulp to heating at a temperature in the range of 105° C. to 150° C., such that the cooked pulp has a pH in the range of from 3.2 to 6.5.

The pineapple pulp used for the purposes of this invention may be derived from any part of the pineapple plant, including the fruit, stem, leaves and root. Most preferably, the pulp is a food-grade by-product of a commercial juicing operation, such as discarded skin or clarifier centrifuge sludge.

The pineapple pulp used for the purposes of this invention may be used as-is, containing residual juice, or may be washed to recover juices prior to extraction. Whether the pulp is washed or unwashed is irrelevant to the effectiveness of the extraction process. There may, however, be implications for the commercial value or ease of disposal of the residual pulp juices.

In our experience, it is not practical to fully wash out all the soluble sugars, etc, prior to extraction because the pineapple pulp tissue represents a substantial diffusional barrier. It would only be feasible to extract the sugars, etc, fully by using a very large wash volume and long contact times, which is not commercially feasible. Therefore, our preference is to extract unwashed pineapple pulp, and to separate the released sugars, etc, from the soluble fiber at a subsequent stage.

On the other hand, the high proportion of sugar and phenolic compounds in the peel juice result in the formation of dark colored compounds during extraction and subsequent processing. The extent of color formation can be reduced by washing the pulp prior to extraction. In this case, the extracted sugars, etc, could be collected as a separate stream, and optionally added to the washed pulp stream.

Depending on the raw material source, the pineapple pulp may require a size reduction treatment so that it may be pumped as a slurry. This may be achieved by any convenient method, such as milling, slicing or shredding. In general, the extraction process becomes more efficient as the particle size is decreased, due to reduced diffusional limitations. However, it is not desirable to reduce the pulp to very fine particles, as it becomes more difficult to separate the solubilised fiber from insoluble residue. The pineapple pulp may have a particle size ranging from 0.5 mm to 50 mm, with best results with a range from 2 mm to 20 mm, most preferably from 5 mm to 10 mm.

The pineapple pulp is admixed with a quantity of water suitable to render it a pumpable slurry. The acceptable ratio of water to pulp ranges from 0:1 to 100:1, although for economic and practical reasons a better range is 0.5:1 to 5:1, with the most preferable range being 1:1 to 2:1.

We have found that the extraction efficiency may be enhanced by the addition of alkali to the water before addition to the pineapple pulp or, alternatively, to the slurry of water and pulp. This can be accomplished using any suitable alkali, including but not limited to sodium hydroxide, potassium hydroxide, barium hydroxide, lithium hydroxide, calcium hydroxide, calcium oxide, ammonia solution or sodium bicarbonate. For a food-grade fiber product, a food-approved alkali is used.

The natural pH of pineapple pulp is about 3.7, mainly due to citric acid and malic acid, the main organic acids in pineapple. Citric acid contains three carboxylic acid groups, while malic acid has two, so the mixture of acids in pineapple pulp has quite a complex dissociation behaviour in the presence of a strong alkali, such as sodium hydroxide. We have found that the pH of a pineapple pulp remains in the acidic region, even after addition of significant amounts of sodium hydroxide. For example, a mixture of equal parts of pineapple pulp and pH 10 NaOH solution reaches an equilibrium pH of 5.9.

We have found that the fiber extraction efficiency is not significantly influenced by using water adjusted to initial pH values ranging from 3 to about 12. This appears to be due to the dissociation properties of the natural acids, which pull the equilibrium pH value down to the range of about 3.4 to about 6.5.

The best results in extracting soluble antioxidant fiber result in a pH in the range of from 3.2 to 6.5 and preferably 3.5 to 5.6. The preferred pH range includes a pH of 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5 and 5.6.

However, we have found that there are disadvantages to adding alkali to the extraction slurry. One disadvantage is that the color of the extracted fiber becomes progressively darker with increasing amounts of added alkali. Also, the added alkali remains in the fiber extract in salt form, and must subsequently be removed, creating a potential waste disposal problem.

Accordingly, we have found it advantageous to avoid the use of alkali altogether, and to extract the pineapple pulp at around the natural pH of 3.7. However, despite the simplicity of extracting at the natural pH, there may be advantages to adding alkali and extracting at higher pH, despite the additional costs involved. For example, extraction pH may be used to control the molecular weight and thus viscosity and proportion of measurable dietary fiber in the product. The extraction pH may also be used to optimize the antioxidant content of the fiber or in response to seasonal variations in fruit quality.

The extraction method involves heating the pineapple pulp to a temperature in the range of 105° C. to 150° C. It is particularly preferred that the pineapple pulp is heated under pressure at a temperature of from 110° C. to 145° C. and most preferable from 120° C. to 140° C. The preferred temperature range includes temperatures of 110° C., 115° C., 120° C., 125° C., 130° C., 135° C., 140° C. and 145° C. The pineapple pulp is preferably heated under pressure, for example in a closed vessel, and the temperature in the range of 120° C. to 140° C.

The period for which the pineapple pulp is heated would generally be at least 30 seconds but for practical purposes a period of at least 3 minutes and less than 5 hours is preferred. The optimum heating period depends on the heating temperature selected, with shorter times needed at higher temperatures. A heating time of between 3 minutes and 10 minutes may be sufficient at 145° C., at 120° C. heating for 1 to 2 hours is preferred, while at 105° C. a period as long as 5 hours may be preferred.

Extraction may be either batch or continuous, and may be effected by direct steam injection, indirect steam heating or microwaving.

While we have found that the heating of pineapple pulp at the chosen pH range enables the preparation of water soluble antioxidant fiber, the yield of water soluble fiber is significantly increased if the pulp is then subject to a process of either ultrasonic or mechanical treatment to facilitate removal of the soluble material from the solids. Ultrasonic treatment may be conducted either during or after the heat treatment process, but is most advantageously conducted by treating the cooked pulp slurry in a continuous manner in a flow-through ultrasonic chamber. One or more passes through such a chamber may be required to achieve the desired degree of solubilisation of the fiber. The softened parenchymal tissue may also be separated from insoluble fibrous residue using mechanical means such as a disc mill, plate refiner, hammer mill or slicer. However, such methods have the disadvantage of reducing the particle size of the insoluble fiber, creating insoluble fines which must subsequently be removed. Ultrasonic treatment is particularly advantageous because it separates the soluble fiber without significantly altering the insoluble residue.

The process of the invention involves separation of the solubilised fiber from insoluble material. A range of separation techniques known in the art may be used to achieve separation of the soluble and insoluble materials. Examples of suitable methods include pressing (e.g. screw press, hydraulic press), filtration (e.g. drum filter, disc filter, basket centrifuge, belt filter), and gravity settling (e.g. hydrocyclone, decanter centrifuge, clarifier centrifuge). Selection of an appropriate method is dependent on the particle size of the pulp slurry and the loading of insoluble fines. Capital cost and waste disposal limitations are also important issues to take into account. For example, a clear solution of solubilised fiber could be produced by filtering the cooked slurry using diatomaceous earth as a filter aid, but disposal of the filter aid may be a problem. A clear solution of solubilised fiber could be produced using a combination of a decanter centrifuge and a clarifying centrifuge, but the capital cost involved is relatively high. Our preferred option is to separate the fiber solution from the pulp using a screw press and to remove fines using a clarifier centrifuge.

We have found that the insoluble fiber component contains antioxidant rich lignin. The insoluble fiber component provided from the process may therefore be useful as an antioxidant where solubility is not a necessary requirement.

The method of the invention may involve concentration of the water soluble fiber component. This could be accomplished using evaporation or a membrane process such as microfiltration, ultrafiltration, nanofiltration or reverse osmosis. The advantage of using microfiltration or ultrafiltration is that such processes allow concentration of the fiber with simultaneous removal of sugars, acids and ash components, thereby producing a purer fiber product which is easier to dry. Suitable microfiltration membranes would have a pore size of between 0.1 micron and 1 micron, preferably between 0.2 micron and 0.45 micron. In our experience, success with microfiltration is dependent on very tight control of transmembrane pressures and, even then, only the highest molecular weight fiber is retained. A higher yield of fiber product can be achieved using ultrafiltration membranes with a molecular weight cutoff in the range of between 1,000 and 100,000, preferably in the range of 10,000 to 20,000.

The method of the invention preferably includes a step of at least partially removing free sugars from the soluble fiber composition for example by diafiltration. This has the advantage of reducing the concentration of sugars to allow the pineapple fiber to be dried to a non-hygroscopic powder. Diafiltration can be most advantageously accomplished using the same type of membranes used during the concentration step. Diafiltration should preferably be conducted to the extent that the ash level of the pineapple fiber product is between 1% and 5% on a dry matter basis. This may be accomplished by using the equivalent of 3 to 5 volume changes of water during batchwise diafiltration, depending on whether or not the pulp was washed before cooking.

In this manner, the water soluble antioxidant fiber may be concentrated to between 20% and 40% w/w, depending on the viscosity of the solution. The concentrated solution may be further pasteurized, if required, and used in this form as a food ingredient.

The concentrated water soluble antioxidant fiber is preferably dried to form a particulate solid. Suitable drying methods include spray drying, freeze drying, drum drying or the like In a further embodiment the invention provides a solid particulate water soluble fiber comprising antioxidant and obtained from pineapple pulp.

A method of the invention may involve addition of additives and stabilisers to prevent browning of the fiber during processing. It is particularly preferred to add sulphur dioxide in the form of sulphur (iv) oxyanines such as HSO₃⁻, SO₃²⁻. Surprisingly we found the addition of sodium metabisulphite to the pineapple pulp prior to autoclaving is sufficient to prevent or substantially reduce brown color formation. The amount of sodium metabisulphite added will preferably be in the range of from 10 to 1,000 ppm and most preferably in the range of from 100 to 300 ppm. We have also found that addition of ascorbic acid to the clarified fiber solution can further prevent oxidative browning during concentration, diafiltration and drying. The amount of ascorbic acid added will preferably be in the range of from 10 to 2,000 ppm. We have found that addition of 500 to 1,000 ppm to the fiber solution is preferable, while further addition of 10 to 50 ppm to the diafiltration water is also advantageous. If ascorbic acid is used during processing, the pH may need to be adjusted accordingly using a suitable alkaline agent, such as sodium hydroxide, potassium hydroxide, barium hydroxide, lithium hydroxide, calcium hydroxide, calcium oxide, ammonia solution or sodium bicarbonate.

A method of the invention may involve additional means of preventing browning, either as an alternative to the use of additives and stabilisers or in addition to such use. Such additional means include processing under an inert atmosphere such nitrogen to prevent oxidation, or use of activated carbon to remove free phenolic compounds from the fiber solution. We have found that activated carbon can successfully be used to remove freely-soluble phenolic compounds from the soluble fiber solution, although care must be taken to select a grade of carbon that maximizes adsorption of phenolics and minimizes adsorption of fiber.

In a further aspect of the invention provides a particulate soluble fiber prepared according to the method of the invention. The soluble fiber product of this invention is comprised of 75% to 99% carbohydrate, 0.5% to 5% lignin, 0.5% to 5% fat, 1% to 10% protein and 1% to 5% ash, on a dry weight basis, and most preferably 80% to 95% carbohydrate, 1.5% to 3.5% lignin, 1% to 4% fat, 2% to 7% protein and 1.5% to 3% ash. The carbohydrate component is comprised of 60% to 90% total dietary fiber (as measured by AOAC Official Method 991.43) and 10% to 40% sugars and oligosaccharides, and preferably 70% to 90% total dietary fiber and most preferably 80% total dietary fiber. The total dietary fiber is comprised of 90% to 100% soluble dietary fiber and 0% to 10% insoluble dietary fiber (as measured by AOAC Official Method 991.43), and preferably 95% to 100% soluble fiber and 0% to 5% insoluble fiber.

The carbohydrate component of the pineapple fiber product of this invention is in one preferred embodiment comprised of 40-80% xylose, 5-25% arabinose, 2-15% galactose, 0.1-15% glucose, 0.1-10% mannose, 0-2% rhamnose/fucose and 5-25% uronic acid, on a mole-percentage basis.

The carbohydrate component of the pineapple fiber product of this invention also contains the phenolic acids, ferulic acid and p-coumaric acid, in a ratio and total amount dependent on the raw material and the method of extraction. Typically, the soluble dietary fiber contains 0.5% to 1% (w/w) total phenolic acids, including 0.005% to 0.02% in freely soluble form, and the remainder covalently bound to the hemicellulose sidechains of the carbohydrate component. The total phenolic acid content has an associated antioxidant activity of at least 50 micromoles Trolox equivalent per gram of fiber, as measured by the ORAC assay of Ou et al [J. Agric. Food Chem. 49 (2001) 4619-4626], and preferably 200-800 micromoles Trolox equivalent per gram of fiber. This antioxidant activity is equivalent to 150-650 mg Vitamin E per gram of fiber.

### BRIEF DESCRIPTION OF THE DRAWINGS

Examples of the invention are described with reference to the attached drawings.

In the drawings:

FIG. 1 is a schematic chart comparing the antioxidant (AOX) content and solubility of fibers from various dietary sources;

FIG. 2 is a graph showing the viscosity response of Composition C of Example 6 at different shear rates as reported in Example 7;

FIG. 3 is a graph showing the variation of viscosity at a shear of 15 sec⁻¹ for different concentrations of Composition C of Example 6 at 25° C. as described in Example 7; and

FIG. 4 is a graph which compares the variation of viscosity with shear for Composition C with commercially available dietary fibers at a concentration of 12 g/L in apple juice as described in Example 10.

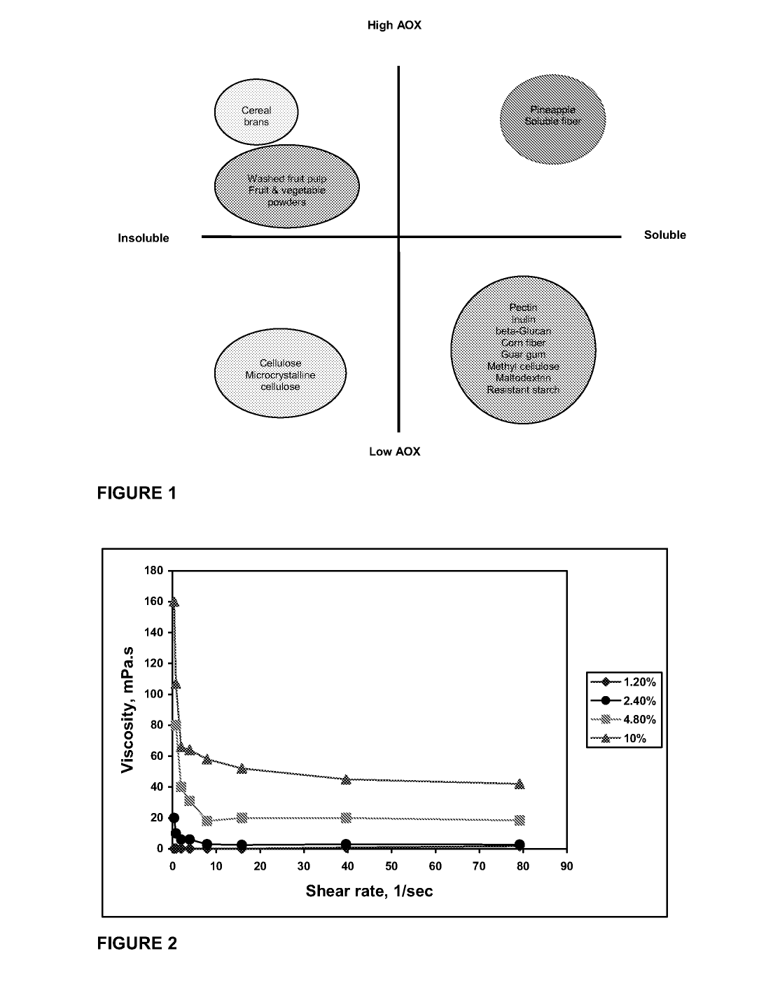

The soluble dietary fiber product of this invention is unique in being the only dietary fiber product available that is rich in both antioxidants and soluble fiber. This is highlighted in FIG. 1, which compares the product of this invention with other commercially available products. This comparison is made by showing how each product is positioned in terms of both antioxidant content and solubility in solution, forming four separate quadrants.

Commercial products which are rich in antioxidants but are poorly soluble are exemplified by cereal brans, and either washed or unwashed fruit and vegetable powders. Products which are highly soluble but lacking antioxidant activity are exemplified by vegetable gums such as pectin, beta-glucan, inulin, guar, methylcellulose and resistant starch. Products which are both insoluble and lacking antioxidant activity are exemplified by the celluloses.

The soluble dietary fiber product of this invention forms a low-viscosity solution in water which is shear-thinning at low shear rates and Newtonian at shear rates above about 10 sec⁻¹. At 25° C., the viscosity is 0.5-1 mPa·s at 1.2% w/v, 3 mPa·s at 2.4% w/v, 20 mPa·s at 4.8% w/v and 40-50 mPa·s at 10% w/v. The solution remains a pourable liquid at a concentration as high as 40% w/v.

In yet another aspect the invention provides a food composition comprising a soluble dietary fiber as described herein. Such foods may include drinks, dairy products, soy and grain milks, soups, baked goods and snack bars, meat products, emulsified edible oils, encapsulated edible oils, instant drinks, instant desserts and soup mixes.

Drink compositions comprising the soluble dietary fiber of this invention may be any suitable beverages such as fruit and vegetable juices, milk drinks, soy milk, rice milk, drinking yoghurt and other acidified dairy drinks.

A preferred embodiment is a fruit or vegetable juice. The particulate soluble fiber material may be added to the beverage by the consumer or alternatively the particulate composition of the invention may be used to formulate storage stable beverages such as fruit and vegetable juice. The soluble fiber product may be used to fortify the dietary fiber content of fruit and vegetable juices to physiologically beneficial levels, allowing food manufacturers to make health claims for the beverage. For example, addition of the soluble fiber product at a concentration to provide 1.5 grams per serve provides “a source of fiber”; 3 grams per serve provides “a good source of fiber”; 6 grams per serve provides “an excellent source of fiber”;

One of the significant advantages of the soluble dietary fiber of the invention is that addition of the product at physiologically beneficial concentrations does not detract from the appearance or taste of natural vegetable or fruit juices. It generally does not unduly affect the viscosity of the beverage or provide the unattractive mouth feel associated with pectin or beta-glucan, or the acrid taste associated with tea and apple polyphenols. It generally does not unduly affect the color of the beverage, providing a neutral-colored cloud effect.

Another significant advantage of the soluble fiber of this invention is that it is stable under the pH and temperature conditions associated with production and storage of shelf-stable fruit and vegetable juices. It provides a stable fiber formulation, unlike inulin which degrades rapidly under such conditions.

Yet another advantage of the soluble fiber of this invention is that it supplements fruit and vegetable beverages with significant additional antioxidant capacity. This is likely to be beneficial in several ways: by helping to preserve the stability of the natural antioxidants present in fruit and vegetable beverages; by helping to protect natural antioxidants such as Vitamins C and E during the digestive process; and by providing a sustained release of antioxidants in the large intestine through progressive microbial hydrolysis.

The soluble dietary fiber of this invention may also be employed in fruit and vegetable beverages in the form of an emulsion with edible oils, to allow fortification of beverages with beneficial ingredients such as omega-3 fatty acids.

A unique advantage of the soluble dietary fiber of this invention is that it can function as both an emulsifier and a natural antioxidant, thereby assisting in the formulation and stabilization of sensitive edible oils in food applications.

Generally the particulate soluble dietary fiber of the invention will be added to the beverage in an amount of at least 0.1 gram per litre and typically no more than 100 grams per litre.

A preferred embodiment is a dairy beverage. The soluble dietary fiber of the invention is compatible with milk and does not cause phase separation or curdling. The soluble dietary fiber may be used as a stabiliser, to fortify the dietary fiber content of the dairy beverage, or as a carrier and stabilizer of emulsified edible oils.

Generally the particulate soluble dietary fiber of the invention will be added to the dairy beverage in an amount of at least 0.1 gram per litre and typically no more than 100 grams per litre. The powdered fiber product would typically be blended with the milk prior to homogenization and pasteurization.

A preferred embodiment is a soy or cereal beverage (e.g. rice milk). Such applications of the soluble dietary fiber of the invention would be in a manner and concentration similar to use in dairy beverages.

A preferred embodiment is in fermented dairy and soy products. The particulate composition of the invention may be used to formulate products such as dairy and soy yoghurts, drinking yoghurts, acidified dairy drinks and cheeses. In such applications, the soluble dietary fiber of this invention may function as a source of dietary fiber, as a stabiliser, as a fat replacement, or as a prebiotic ingredient. A significant advantage of the dietary fiber of this invention in such applications is that it has no adverse impact on texture or color, while smoothing the taste profile associated with the lactic acid in such fermented products.

Generally the particulate soluble dietary fiber of the invention will be used in such applications in an amount of at least 0.1 gram per litre and typically no more than 100 grams per litre.

In some embodiments the dietary fiber is used in soup products. In such applications, the soluble dietary fiber of this invention may function as a source of dietary fiber, to supplement the low fiber content associated with most vegetable soups. A significant advantage of the dietary fiber of this invention in such applications is that it has no adverse impact on texture or color.

Generally the particulate soluble dietary fiber of the invention will be used in such applications in an amount of at least 0.1 gram per litre and typically no more than 100 grams per litre.

In some embodiments the dietary fiber is used in baked goods. The particulate composition of the invention may be used to formulate products such as bread, breakfast cereals, cookies, muffins, etc. A significant advantage of the dietary fiber of this invention in such applications is that it provides a high antioxidant capacity which survives baking temperatures. In such applications, the soluble dietary fiber of this invention may provide some of the nutritional benefits associated with fruit without significantly affecting the color, flavour or texture of the baked product. The soluble dietary fiber of this invention may also be used in the form of an emulsifying or encapsulating agent, to facilitate the formulation and stabilization of sensitive edible oils in baked goods.

Generally the particulate soluble dietary fiber of the invention will be used in such applications in an amount of at least 0.1 gram per kilogram and typically no more than 100 grams per kilogram.

In some embodiments the dietary fiber is used in snack bars such as those incorporating one or more of cereal products, seeds and fruits. In such applications, the soluble dietary fiber of this invention may provide some of the nutritional benefits associated with fruit without significantly affecting the color, flavour or texture of the product.

Generally the particulate soluble dietary fiber of the invention will be used in such applications in an amount of at least 0.1 gram per kilogram and typically no more than 100 grams per kilogram.

Another preferred embodiment involves the use of the dietary fiber in meat products, such as fresh fish and poultry, and processed meats. In such foods the soluble dietary fiber of this invention may act as a natural antioxidant to prevent the onset of rancidity.

Generally the particulate soluble dietary fiber of the invention will be used in such applications in an amount of at least 0.1 gram per kilogram and typically no more than 100 grams per kilogram.

Yet another embodiment involves the use of the dietary fiber in dry premix products. Such applications include instant drinks, dessert mixes and soup mixes. In such foods the soluble dietary fiber may function as a source of dietary fiber, a natural clouding agent, an encapsulating agent or as natural antioxidant to prevent the onset of rancidity.

Generally the particulate soluble dietary fiber of the invention will be used in such applications in an amount of at least 0.1 gram per kilogram and typically no more than 100 grams per kilogram.

In some embodiments the dietary fiber is used as an encapsulating agent for edible oils. Such applications may include oils such as fish oils, microalgal oils, single-cell omega-3 fatty acids, essential oils, flavours and aromas. In such foods the soluble dietary fiber of this invention may function as an emulsifier, an encapsulating agent and as a natural antioxidant to prevent the onset of rancidity.

Generally the particulate soluble dietary fiber of the invention will be used in such applications in an amount of at least 100 gram per kilogram and typically no more than 950 grams per kilogram.

In yet another aspect the invention provides a food supplement comprising a soluble dietary fiber as described herein.

In yet another aspect the invention provides a cosmetic composition comprising a soluble dietary fiber as described herein.

In yet another aspect the invention provides a pharmaceutical composition comprising a soluble dietary fiber as described herein.

The invention will now be described with reference to the following examples. It is to be understood that the examples are provided by way of illustration of the invention and that they are in no way limiting to the scope of the invention.

### EXAMPLES

### Comparison 1

Pineapple core has been used as a source of a bulking agent, useful as a partial replacement for high-caloric ingredients such as flour, fat and/or sugar [Altomare et al U.S. Pat. No. 4,431,677, Feb. 14, 1984]. This process involved washing chopped pineapple core with water, then alcohol, followed by drying and milling. The product was reported to contain 30-40% cellulose, 25-35% hemicellulose, 3-10% pectin, 15-25% lignin 2-8% protein and 1-5% ash. The resulting fiber product is essentially insoluble, forming a turbid suspension in water and settling out upon standing [Prakongpan et al J. Food Sci. 67 (2002) 1308-1313].

Similarly, we blanched pineapple pulp at 90° C. for 1 minute, then washed it three times with 2 mass equivalents of water at 50° C., followed by freeze-drying. The dried pulp was reduced to a fine powder using a cross-beater mill, and sieved to a screen size of 65-200 mesh.

This powder was added to commercial apple juice at a concentration of 12 g/L. The apple juice was first heated by microwaving to 60° C., then the powder was dispersed into the juice using a kitchen blender. The juice mixture was refrigerated and stored for 1 week. After this time, the pineapple powder had completely settled as a thick sludge at the bottom of the container.

### Comparison 2

Chan & Moy [J. Food Sci. 42 (1977) 1451-1453] report the extraction of hemicellulose-B from centrifuge sludge collected from a commercial pineapple juice operation. In their procedure the pulp is washed with acetone, then boiling water, followed by extraction with 4N sodium hydroxide at room temperature under nitrogen for 24 hours. Under this type of conditions Hartley 1978 reported that the associated phenols are removed.

Similarly, we extracted blanched, washed pineapple pulp under the same conditions, then neutralized the mixture with nitric acid to pH 8. The extracted fiber was separated from insoluble residue using a filter cloth. The filtrate was concentrated on a 10 kD cut-off Amicon polysulfone ultrafiltration membrane, and then diafiltered batchwise with five volume changes of water to remove residual sugars and salts. The resulting washed fiber solution was freeze-dried.

The dried pineapple fiber was added to apple juice in the manner described in Comparison 1. The resulting mixture had a turbid grey-green color which would not be commercially acceptable. After 1 week refrigerated storage, a thick, loose deposit had formed at the bottom of the container, but the bulk of the juice retained the grey-green color.

### Example 1

Pineapple pulp was blanched at 90° C. for 1 minute, then washed three times with 2 volumes of water at 50° C. The blanched, washed pulp was suspended in 2 mass equivalents of water, then autoclaved at 120° C. for 1 hour. After cooling to approximately 50° C., the mixture was filtered and pressed manually using calico cloth, then centrifuged to remove fines. The pH of the filtrate was 3.7. The clarified filtrate was concentrated on a 3 kD cut-off Amicon cellulose acetate ultrafiltration membrane, and then diafiltered batchwise with five volume changes of water to remove residual sugars and salts. The resulting washed fiber solution was freeze-dried.

The dried pineapple fiber was added to apple juice in the manner described in Comparison 1. The fiber imparted a pale golden-brown color to the juice and a slight haze. After storage under refrigeration for 1 week there was no observable precipitation.

The dietary fiber content of the dried pineapple fiber was analysed at BRI Research Pty Ltd (Sydney, NSW, Australia) using AOAC Official Method 991.43. The total dietary fiber content was found to be 64.4%, comprising 2.1% insoluble fiber and 62.3% soluble fiber. Thus, the dietary fiber extracted from pineapple at pH 3.7 was >95% soluble fiber.

The antioxidant activity of the dried fiber was analysed at Southern Cross University (Lismore, NSW, Australia), using the Oxygen Radical Absorbance Capacity (ORAC) assay [Ou et al J. Agric. Food Chem. 49 (2001) 4619-4626]. The phenolic compounds present were detected by HPLC and quantified as ferulic acid. “Free” phenolic compounds were extracted using methanol/acetone/water (7:7:6 v/v/v). “Total” phenolic compounds, including fiber-bound phenolics, were liberated by digestion in 2 N sodium hydroxide under nitrogen at room temperature then extraction with diethyl ether/ethyl acetate (1:1 v/v), as described by Abdel-Aal et al [J. Agric. Food Chem. 49 (2001) 3559-3566].

The analysis of the extracted pineapple fiber is shown in Table 1, as well as data for the samples produced as Comparison 1 and Comparison 2.

TABLE 1

| | |
|---|---|
| Phenolics | ORAC value |
| (% w/w) | (μmol TE/g) |

| Sample | Free | Total | Free | Total |
|---|---|---|---|---|
| Comparison 1 | 0.065 | 0.988 | 7.0 | 214.0 |
| Comparison 2 | 0 | 0 | 0.2 | 5.9 |
| Example 1 | 0.028 | 0.796 | 65.9 | 295.9 |

This data shows that, in the washed pineapple pulp of Comparison 1, the majority of phenolic compounds are present in a bound form associated with the cell wall polysaccharides. The proportion of free phenolics is low, presumably due to the effectiveness of the washing procedure. The alkali-extracted pineapple fiber of Comparison 2 contained no detectable phenolics and had essentially no antioxidant activity. In contrast, the pineapple fiber produced by autoclave extraction at pH 3.7 retained a high level of fiber-bound antioxidant activity.

The antioxidant activity of the samples is expressed as micromoles Trolox Equivalent per gram. Vitamin E (alpha-tocopherol) has half the ORAC value of Trolox [Huang et al J. Agric. Food Chem. 50 (2002) 1815-1821] and has a molecular weight of 430.7 g/mol. Hence, the total antioxidant activity of the fiber of Example 1 may be expressed as being equivalent to 250 mg Vitamin E per gram. Such a high antioxidant value is unprecedented for a soluble dietary fiber.

This is the first time that the phenolic content and associated antioxidant activity of both the “free” and “total” fractions have been reported for pineapple. We have found that washed pineapple pulp contains a relatively high proportion of wall-bound antioxidant phenolic compounds, which have not hitherto been properly accounted for. This helps to explain the relatively low antioxidant activity attributed to pineapple, since the prior reports have only been based on readily-extractable “free” phenolic compounds.

This work also shows that the pineapple hemicellulose B extracted by Chan & Moy 1977 would have had negligible antioxidant activity. Therefore, for the first time, we have shown that it is possible to produce an antioxidant-rich soluble dietary fiber from pineapple pulp using autoclave extraction at pH 3.7. This was surprising and not anticipated by the prior art, which taught that mild acidic extraction of arabinoxylan hemicelluloses would strip off feruloylated arabinose side-chains and thus result in a fiber with little or no antioxidant activity.

### Example 2

### Effect of Extraction pH

A series of bench-scale extraction trials were conducted to evaluate the effect of extraction conditions on yield and antioxidant activity. Pineapple pulp was mixed with 2 mass equivalents of water which had been adjusted to a pH in the range of 2 to 12.2 with the addition of either nitric acid or sodium hydroxide. The pulp slurry was then autoclaved at 120° C. for 1 hour, then cooled to 50° C. and filtered through cheesecloth. The filtrate was precipitated with 4 volumes of chilled isopropanol and refrigerated overnight. The mixture was then centrifuged, the supernatant decanted off, and the pellet dried at 60° C. overnight. The yield of alcohol-insoluble solids was expressed as a percentage of the initial fresh weight of unwashed pulp.

Larger samples were prepared by autoclaving larger batches of material and recovering the fiber product by ultrafiltration, diafiltration and freeze-drying, in the manner described in Example 1. Fiber samples were analysed for phenolic content and antioxidant activity at Southern Cross University (Lismore. NSW, Australia).

The results obtained are shown in Table 2.

TABLE 2

| | | | Viscosity, | | | |
|---|---|---|---|---|---|---|
| | Post- | | mPa · s | | | |
| Initial | extraction | Yield (% | (10%, | ORAC value (μmol TE/g) | | |
| pH | pH | Fresh wt) | 20° C.) | Free | Total | Bound |
| 2 | 3.2 | 0.4 | | 0.3 | 10.4 | 10.1 |
| 3 | 3.5 | 0.36 | | 9.6 | 65.1 | 55.5 |
| 3.7 | 3.7 | 0.33 | 60 | 23.5 | 432.3 | 408.8 |
| 6 | 4.0 | 0.28 | 20-30 | | | |
| 8 | 4.1 | 0.29 | 20-30 | 32.5 | 444.7 | 412.2 |
| 11 | 4.3 | 0.30 | 20-30 | 46.1 | 426.5 | 380.4 |
| 12 | 5.3 | 0.29 | | 33.5 | 294.2 | 260.7 |
| 12.2 | 6.9 | 0.24 | | 62.2 | 248.4 | 186.2 |

It was found that the color of the autoclaved slurry varied with the pH. At a final pH of 2, the slurry developed a strong rust-red color, suggestive of liberation and oxidation of phenolic compounds. The color was closest to the starting appearance at a final pH of 3, and became progressively browner as the pH increased.

The yield of soluble fiber was found to be highest at a pH of 3 or lower. However, at a final pH less than 3.7, the natural pH of pineapple, the content of antioxidant phenolic compounds is substantially lost. While the pH 3 sample had a better color than pH 3.7, the antioxidant activity of the fiber was significantly lower.

When the pulp was autoclaved at the natural pH, it was found that the yield, antioxidant activity and viscosity of the fiber were maximal. This suggests that minimum damage to the fiber occurs under these conditions.

Extraction at a final pH higher than 3.7 causes a significant loss of viscosity. Presumably, hydrolysis of the fiber under such conditions results in reduction of molecular weight and reduced efficiency of recovery by alcohol precipitation. The yield of recovered fiber did not alter substantially until the final pH reached 6.9, at which point a slight decrease was noted. However, the fiber developed an increasingly dark brown color as the pH was increased. This corresponded to a fall in the bound antioxidant content at final pH values greater than 4.1. Presumably, the released phenolic compounds became oxidized to a brown color and were adsorbed to the fiber through hydrophobic interactions.

It was a great surprise to find that the antioxidant activity was the highest in the pH 3.7 autoclave extract. It is generally believed that extraction under such “mild acidic” conditions will cleave the feruloylated arabinose side-chains, which should result in loss of antioxidant activity. Instead, these results suggest that, with pineapple pulp at 120° C., loss of sidechains occurs at pH values less than 3.7, while cleavage of the backbone with loss of molecular weight occurs at pH values greater than pH 4.0.

From the available results, it appears that the optimum extraction conditions are at a final pH either at or slightly higher than the natural pH. At this stage, the evidence suggests that the highest antioxidant activities are obtained over a final pH range of from 3.7 to about 4.1.

### Example 3

### Effect of Extraction Time and Temperature

Pineapple pulp was blanched at 90° C. for 1 minute, then washed three times with 2 mass equivalents of water at 50° C. The blanched, washed pulp was suspended in 2 mass equivalents of water, then autoclaved using a range of different times and temperatures. After cooling to approximately 50° C., the mixture was filtered and pressed manually using calico cloth, then centrifuged to remove fines. The fiber component was recovered by ultrafiltration, diafiltration and freeze-drying, in the manner described in Example 1. Fiber samples were analysed for phenolic content and antioxidant activity at Southern Cross University (Lismore. NSW, Australia). Total dietary fiber content of the samples was analysed by BRI Research Pty Ltd (Sydney, NSW, Australia) using AOAC Official Method 991.43.

The effect of autoclaving time and temperature on extraction yield and antioxidant content of fiber is shown in Table 3.

TABLE 3

| | | | ANTIOXIDANT CONTENT | | |
|---|---|---|---|---|---|
| | YIELD % | TOTAL | Free | Total | Bound |
| EXTRACTION | Fresh | DIETARY | μmol | μmol | μmol |
| CONDITIONS | Weight | FIBER % | TE/g | TE/g | TE/g |
| 120° C., 60 min | 0.33 | 57.0 | 14.9 | 342.5 | 327.6 |
| 130° C., 20 min | 0.26 | 54.5 | 15.9 | 446.9 | 431.0 |
| 130° C., 40 min | 0.34 | 52.0 | 20.4 | 651.7 | 631.3 |
| 140° C., 20 min | 0.25 | 50.5 | 23.4 | 825.3 | 801.9 |
| 145° C., 5 min | 0.23 | 55.1 | 12.6 | 330.2 | 317.6 |
| 145° C., 15 min | 0.29 | 31.4 | 64.7 | 315.9 | 251.2 |
| 145° C., 5 min | 0.33 | 53.2 | 40.0 | 385.8 | 345.8 |
| then 95° C., | | | | | |
| 60 min | | | | | |

These results show that the yield of soluble fiber and the antioxidant content are influenced by both the time and temperature of extraction. Higher temperatures and times tend to increase the antioxidant activity of the fiber while reducing the total dietary fiber content. Extraction at 145° C. for 15 minutes caused a reduction in both total dietary fiber content and antioxidant activity, suggesting that the fiber is degraded under these conditions.

The data also suggests that there is a diffusion-limited component to the extraction reaction. For example, extraction at either 120° C. for 60 minutes or 130° C. for 40 minutes resulted in similar yields of fiber. However, extraction at higher temperatures for shorter time caused reductions in both yield and dietary fiber content. This suggests that effective extraction requires both sufficient temperature to weaken the tissue structure and sufficient time for solubilized fiber to diffuse out of the tissue matrix.

This concept was tested by exposing the peel to a short period of high temperature (145° C. for 5 minutes) followed by an extended period at a low temperature (95° C. for 60 minutes). On its own, the high temperature treatment would be expected to liberate only about 70% of the maximum yield, while treatment at 95° C. is insufficient to extract any soluble fiber at all. However, it was found that the combination treatment was as effective as the standard extraction conditions (120° C., 60 min).

### Example 4

### Preliminary Evaluation of Ultrasonic Treatment

Pineapple pulp was blanched at 90° C. for 1 minute, then washed three times with 2 mass equivalents of water at 50° C. The blanched, washed pulp was suspended in an equal mass of water, then subjected to three alternative treatments: (a) no heat treatment; (b) boiling at 100° C. for 1 hour; or (c) autoclaving at 120° C. for 1 hour. The three samples were then subjected to ultrasonic treatment for 1 minute each. Ultrasonic energy was delivered using a Hielscher 1 kW Model UIP1000 with a 22 mm focussed sonotrode. The ultrasonic unit operated at 18 kHz and delivered approximately 0.4 kW process energy.

After ultrasonic treatment, the three samples were evaluated both visually and in terms of how easily the liquid could be separated from solids by squeezing in a calico cloth. The results are shown in Table 4.

TABLE 4

| Heat pre-treatment of sample | Effect of ultrasonication |
|---|---|
| None | No apparent breakdown of pulp |
| | structure. |
| 100° C. for 1 hour | No apparent breakdown of pulp |
| | structure. |
| | No improvement in liquids separation. |
| 120° C. for 1 hour | Significant increase in turbidity of |
| | liquid phase. |
| | Pressing of solids was easier, with |
| | more liquid recovered. |
| | Yield of alcohol-insoluble solids |
| | increased by 49%. |

Ultrasonic treatment is typically used to disrupt cell walls and facilitate extraction. However, we found that unprocessed pineapple pulp was highly resistant to ultrasonic treatment over practical time frames (1-5 minutes at bench scale). Even cooking at 100° C. for 1 hour was insufficient to soften the pulp tissue structure sufficiently to allow breakdown by subsequent ultrasonic treatment. However, autoclaving the pulp at 120° C. for 1 hour, followed by ultrasonic treatment proved to be more effective than autoclave treatment alone.

We were surprised to discover that recovery of the solubilised fiber was significantly enhanced by subjecting the autoclaved pulp to ultrasonic treatment. Visually, it could be seen that the insoluble fiber residue was cleaner, with less residual parenchymal tissue attached. Functionally, this meant that the pulp was easier to press, with greater recovery of press liquid and a higher proportion of soluble fiber. The amount of recovered alcohol-insoluble solids was increased by 49%.

### Example 5

### Mechanical Treatment

A preliminary experiment involved blending autoclaved pulp slurry in a domestic food processor. It was found that this treatment reduced the average particle size of the slurry and created a high proportion of fine insoluble particles. These fine particles clogged the filter cloth, making it difficult to separate the soluble fiber extract from the insoluble residue. Such particles can be removed by centrifugation, but increasing the proportion of such particles at commercial scale would reduce the centrifuge throughput or, alternatively, increase the size of centrifuge needed to maintain a given throughput. Hence, creation of fines through rough mechanical treatment increases the costs associated with subsequent downstream processing.

### Example 6

### Pilot-Scale Trial (Including Ultrasonic Treatment)

A pilot-scale trial of the pineapple pulp extraction process was undertaken at Food Science Australia (Werribee, Victoria, Australia).

In this trial, 200 kg frozen pineapple pulp was reduced to a particle size of roughly 1-5 mm in a bowl cutter. The milled pulp was mixed with water (pH 5.5) in 1:1 mass ratio and sealed in 3.5 litre cans. The pH of the mixture was 3.9. The cans were retorted at 120° C. for 3 hours. The temperature inside the cans was at 120° C. for approximately 1.5 hours. The cans were cooled and the contents decanted into a holding tank. The pH of the autoclaved mixture was 3.7.

Initial trials with a Hielscher 8 kW ultrasonic unit involved pumping some of the pulp slurry through the ultrasonic chamber at two different flowrates (12 litres/min and 25 litres/min) for 1 and 2 passes. In each case, a process power input of 3.7 kW was delivered in the ultrasonic chamber.

Samples of treated pulp were filtered by passing through calico cloth, then centrifuged to remove fines. The clarified filtrate was concentrated on a 3 kD cut-off Amicon cellulose acetate ultrafiltration membrane, and then diafiltered batchwise with five volume changes of water to remove residual sugars and salts. The resulting washed fiber solution was freeze-dried. The yield of recovered fiber is shown in Table 5.

TABLE 5

| | Yield |
|---|---|
| Ultrasonic treatment | (% fresh wt unwashed pulp) |
| None | 0.20 |
| 6 L/min, 1 pass | 0.27 |
| 12 L/min, 1 pass | 0.24 |
| 12 L/min, 2 passes | 0.27 |

In this pilot-scale evaluation, it was found that the yield of recovered fiber could be increased by 35% by ultrasonic treatment using either 1 pass at 6 L/min or 2 passes at 12 L/min. In contrast, the preliminary laboratory-scale evaluation (described in Example 4) showed that ultrasonic treatment could increase the yield of fiber by 49%. This difference may be attributed to losses of fiber in the polarized gel layer formed on the ultrafiltration membrane, which would be lower at a larger scale of operation.

Following preparation of these samples for analysis, the bulk of the pulp slurry was passed twice through the ultrasonic chamber at 12 L/min at a power input of 3.7 kW. The treated slurry was then filtered through cheesecloth and clarified using a Westfalia disc-stack centrifuge. The brown-colored extract was then concentrated 10-fold using a 10 kD cut-off Koch polysulfone ultrafiltration membrane. The concentrated fiber was washed by diafiltering with 12 volumes of water. It was found that all the sugar was removed after 5 volumes.

Composition A was prepared by freeze-drying a small sample of the concentrate.

Composition B was prepared by spray drying the concentrate using a Niro Production Minor dryer fitted with a rotary atomizer, with an air inlet temperature of 180° C. air inlet and an air outlet temperature of 85° C. The liquid was very low viscosity and easy to dry, with essentially complete recovery. In this manner, 150 g of a non-hygroscopic powder was produced. The moisture content of Composition B was 9%, measured by drying overnight at 110° C.

The phenolic content and antioxidant activity of the two samples was determined at Southern Cross University (Lismore, NSW, Australia), as shown in Table 6.

TABLE 6

| | Ferulic acid (% w/w) | | Phenolics (% w/w) | | ORAC value (μmol TE/g) | |
|---|---|---|---|---|---|---|
| Sample | Free | Total | Free | Total | Free | Total |
| Composition A | n.d. | 4.84 | 0.008 | 5.66 | 7.5 | 389.8 |
| Composition B | n.d. | 5.27 | 0.010 | 5.91 | 9.0 | 464.4 |

These results show that the antioxidant properties of the pineapple fiber were not damaged during the spray drying process.

The dietary fiber content of Composition B was analysed using AOAC Official Method 991.43. The total dietary fiber content was found to be 68.7%, comprising 0.2% insoluble fiber and 68.4% soluble fiber. Thus, the pineapple fiber extracted in the pilot plant was >99% soluble fiber.

### Example 7

### Pilot-Scale Trial (Including Screw Press Treatment)

A second pilot-scale trial was undertaken to evaluate the potential of using a screw press to separate soluble fiber from extracted pulp, rather than ultrasonic treatment.

Pineapple pulp (700 kg) was prepared and extracted at Food Science Australia (Werribee, Victoria, Australia) in the manner described in Example 6. Following retorting, the cans were transported to another pilot plant at Albright & Wilson (Australia) Ltd (Yarraville, Victoria, Australia). Liquid was pressed from the extracted pulp using a Vincent model VP-6 screw press. The soluble fiber solution was clarified by passing it through a Westfalia model SB7-01-076 disc-stack centrifuge, followed by a 5 μm polishing filter cartridge. The fiber solution was concentrated using a 30 m² Koch polysulfone ultrafiltration membrane with a 20 kD cut-off, then diafiltered with 5 volume changes of RO-purified water. The purified fiber solution was spray dried using a Niro Production Minor dryer operated with an air inlet temperature of 190° C. and an air outlet temperature of 80° C.

This process resulted in the production of 6.96 kg of a non-hygroscopic, tan-colored powder (Composition C), representing a yield of 1% of fiber from the original pineapple pulp. On the basis of the results of Example 6, only about 2-3 kg of powder was expected. This indicated that the mechanical forces involved in screw pressing are much more effective than the combined use of ultrasonic treatment and manual pressing. Thus, when a screw press is used, there is no advantage to be gained from ultrasonic treatment.

Total dietary fiber content of Composition C was analysed by BRI Research Pty Ltd (Sydney, NSW, Australia) using AOAC Official Method 991.43. The total dietary fiber content was found to be 75.5%, comprising 0% insoluble fiber and 75.5% soluble fiber. Thus, the dietary fiber extracted in this pilot plant trial was comprised of 100% soluble fiber.

Proximate analysis of Composition C was done by Dairy Technical Services Ltd (Kensington, Victoria, Australia), yielding data on fat, protein, ash and moisture content. Carbohydrate content (including lignin) was estimated by difference.

Analysis of the composition of the carbohydrate fraction was done at the School of Botany, University of Melbourne (Parkville, Victoria, Australia). Monosaccharide composition was determined using sulphuric acid hydrolysis and TFA hydrolysis, according to the methods of Albersheim et al Carbohydr. Res. 5 (1967) 340-345, Blakeney et al Carbohydr. Res 113 (1983) 291-299 and Saeman et al Methods Carbohydr. 3 (1983) 54-69. Uronic acid was determined by a modified colorimetric procedure (Fillisetti-Cozzi & Carpita Analyt. Biochem. 197 (1991) 157-162) using Glucuronic acid as a standard. Lignin analysis was conducted according to the Klason method.

The combined results of the chemical analysis of Composition C are shown in Table 7.

TABLE 7

| Component | % w/w | Mol % | % w/w |
|---|---|---|---|
| Carbohydrate | 87.2 | | |
| Arabinose | | 5.9 | 4.9 |
| Xylose | | 78.3 | 65.5 |
| Mannose | | 0.4 | 0.4 |
| Galactose | | 2.2 | 2.2 |
| Glucose | | 0.4 | 0.4 |
| Rhamnose/Fucose | | — | 0 |
| Uronic acid | | 12.8 | 13.8 |
| Lignin | 1.8 | | 1.8 |
| Fat | 0.9 | | 0.9 |
| Protein | 2.2 | | 2.2 |
| Ash | 1.5 | | 1.5 |
| Moisture | 6.4 | | 6.4 |

The hemicellulose fraction of pineapple is known to be composed mainly of glucuronoarabinoxylan along with xyloglucans and small amounts of glucomannans (Smith and Harris. Plant Physiol. 107 (1995) 1399-1409). The results in Table 7 suggest that the soluble fiber fraction of Composition C is comprised of about 83% hemicellulose and 16% pectin.

The phenolic content and antioxidant activity of Composition C was determined at Southern Cross University (Lismore, NSW, Australia), as shown in Table 8.

TABLE 8

| | Ferulic acid (% w/w) | | Phenolics (% w/w) | | ORAC value (μmol TE/g) | |
|---|---|---|---|---|---|---|
| Sample | Free | Total | Free | Total | Free | Total |
| Composition C | 0.001 | 4.4 | 0.04 | 5.7 | 38.5 | 464.6 |

This data indicates that ferulic acid is the main phenolic compound associated with the soluble fiber in Composition C. The antioxidant activity of Composition C is consistent with the value obtained in the previous pilot plant batch (Composition B of Example 6).

The viscosity of Composition C as a function of concentration in water at 25° C. was measured using a Brookfield DVII+ viscometer with a SC4-18/13R small volume spindle. FIG. 2 shows that pineapple fiber is strongly shear-thinning at shear rates below about 10 sec⁻¹, and essentially Newtonian at higher shear rates.

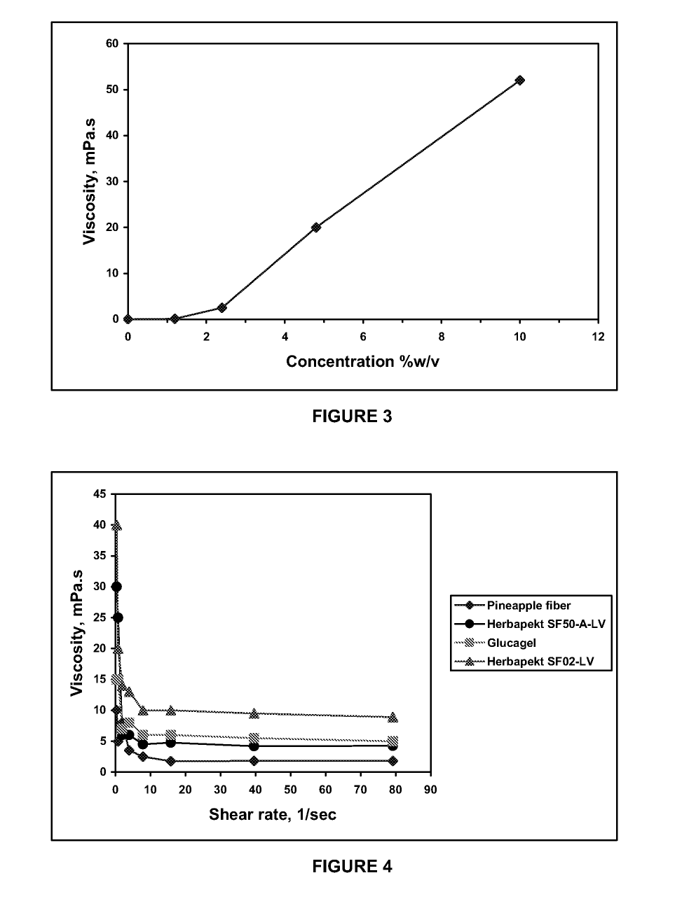

To illustrate this data in more detail, the viscosity measured at a shear rate of 15 sec⁻¹ is shown in FIG. 3 as a function of concentration.

The viscosity of Composition C increases sharply with increasing concentration. Nevertheless, the concentration is very low compared with most other natural vegetable gums, with the exception of gum Arabic. Even at a concentration of 10%, the viscosity is only 50 mPa·s, which is equivalent to a 30% solution of Gum Arabic at the same shear rate [Islam et al Food Hydrocolloids 11 (1997) 493-505].

A 40% concentration of Composition C remains a pourable liquid, which suggests that it may be feasible to provide commercial quantities of the soluble fiber product of this invention in the form of a liquid concentrate. This may facilitate easier incorporation of the fiber product into liquid food products.

### Example 8

### Reduction of Color

In our experience, the main barrier to practical application of the soluble fiber of this invention is the color which develops during extraction and subsequent processing. We have found that color develops by two separate mechanisms. During the high temperature extraction process, brown Maillard pigments form as the result of reaction of sugars with proteins and amino acids. During subsequent downstream processing, rust-colored coloration develops due to oxidization of freely-soluble phenolic compounds.

We have found that, once formed, the pigment compounds are not readily separated from the fiber, either by diafiltration or by alcohol precipitation. This suggests that the pigmented compounds are relatively hydrophobic, and tend to adsorb to the fiber in solution. In our experience, it is better to prevent the color developing than to try to clean up the fiber later.

Nonenzymatic browning reactions occur commonly during the cooking of foods. Nonenzymic browning reactions involve either the heat-induced decomposition reaction of sugars (without amine participation) that is called “caramelization”, or the reaction in which the carbonyl groups of acyclic sugars are condensed with the basic amino groups of proteins, peptides and amino acids, known as the Maillard reaction. The brown pigments formed in the Maillard reaction are known as melanoidins. The formation of melanoidins is known to be inhibited by SO₂, through reactions of precursors of melanoidins with sulphite and hydrogen sulphite ions, to form products with a reduced browning potential (Wedzicha & Kaputo Int. J. Food Sci. Technol. 22 (1987) 643-651].

Formation of melanoidins during autoclave extraction can be inhibited by addition of sodium metabisulphite to the pineapple pulp slurry at a concentration of 10 ppm to 1,000 ppm, preferably 50 ppm to 500 ppm and most preferably 100 ppm, 200 ppm or 300 ppm. The advantage of sodium metabisulphite is that it is relatively inexpensive and does not alter the pH of the slurry. Potassium metabisulphite is also suitable, although more expensive. Sulfur dioxide may also be used, although this will require addition of alkali to neutralize the resulting pH fall.

Oxidation of freely-soluble phenolics can be prevented in three different ways, either alone or in combination: by conducting all processing in an oxygen-free environment under nitrogen; through the use of sacrificial antioxidants during processing, or by removal of free phenolics altogether.

At a commercial scale, it is expected that oxidation may be best controlled by minimizing the time between juicing the pineapple fruit and drying the soluble fiber product. Processing under a nitrogen atmosphere, to minimize contact with oxygen, would also be beneficial. However, neither of these options are easily done at laboratory and pilot scales.

For small-scale work, we have found that oxidization of freely-soluble phenolics can be prevented by addition of ascorbic acid to the clarified fiber solution. The amount of ascorbic acid added should be in the range of from 10 ppm to 2,000 ppm, preferably 500 ppm to 1,000 ppm. If ascorbic acid is used during processing, the pH may need to be adjusted accordingly using a suitable alkaline agent, such as sodium hydroxide, potassium hydroxide, barium hydroxide, lithium hydroxide, calcium hydroxide, calcium oxide, ammonia solution or sodium bicarbonate. Alternatively, sodium ascorbate or potassium ascorbate may also be used.

It may also be advantageous to add ascorbic acid to the diafiltration water, to compensate for loss of ascorbic acid during diafiltration. In this case, a concentration of 10 ppm to 100 ppm is advantageous, preferably 20 ppm to 50 ppm.

The most desirable way to prevent oxidation of freely-soluble phenolics is to remove them from solution altogether. This can be accomplished by treating the fiber solution with activated carbon as soon as possible after the clarification step, to minimize the opportunity for color development.

For example, pineapple pulp was blanched at 90° C. for 1 minute, then washed three times with 2 mass equivalents of water at 50° C. The blanched, washed pulp was suspended in 2 mass equivalents of water, then autoclaved at 120° C. for 1 hour. After cooling to approximately 50° C., the mixture was filtered and pressed manually using calico cloth. The resulting solution was recirculated from an open tank with a peristaltic pump for 4 hours, to simulate the effects of extended processing in the presence of atmospheric air. During this time, it was observed that the color of the fiber solution became progressively browner.

The colored fiber solution was used to evaluate the effectiveness of a series of activated carbon samples in removal of phenolic compounds. All the activated carbon samples were provided by Cuno Pacific, as part of the ZetaPlus range of products. Each sample was provided as a “Biocap”, in the form of a 40 mm×6 mm disc. Each activated carbon sample was used to treat a 200 mL volume of colored fiber solution. The effectiveness of color removal was assessed both visually and by measuring the absorbance at wavelengths spanning 300 nm to 400 nm. The relative efficacy of the various samples was ranked on a scale from 0 to 5 for color reduction, as shown in Table 9.

TABLE 9

| Type | Efficacy ranking |
|---|---|
| R54S | 4 |
| R55SP | 4 |
| R32S | 3 |
| R12S | 3 |
| R52S | 2 |
| A51SP | 2 |
| A53SP | 4 |
| AR13S | 4 |
| AR11S | 3 |

These results suggest that activated carbon can be used to remove color-forming phenolics from the soluble fiber solution, but that selection of a suitable grade is crucial to success.

### Example 9

### Pilot-Scale Trial (Continuous Cooking)

A third pilot-scale trial was undertaken to evaluate the potential of using a continuous cooking process, rather than batch retorting. Cooking was accomplished by direct steam injection, using a custom-built test facility at Gold Peg International Pty Ltd (Moorabbin, Victoria, Australia).

700 kg of semi-frozen pineapple pulp was milled using a Reitz pilot-scale hammer mill. The pulp was mixed with 2 parts of tap water containing 500 ppm sodium metabisulphite, then cooked at 145° C. for 3 minutes. The temperature was reduced to 50° C., then the cooked pulp slurry was sealed into 200 litre drums for transport to the pilot plant at Albright & Wilson (Australia) Ltd (Yarraville, Victoria, Australia). There, soluble fiber was recovered in the manner described in Example 7.

This process resulted in recovery of 3.4 kg of spray-dried powder (Composition D), representing a yield of 0.49%. This was only half the yield from the retorting process of Example 7, which is consistent with the data in Table 3.

In addition, a sample was collected of the insoluble sludge discharged from the clarifier centrifuge. This material was washed repeatedly with RO-purified water to remove free sugars and water-soluble dietary fiber, and then freeze-dried. The resulting powder, Composition E, represents the insoluble fiber component that may be extracted from pineapple pulp.

Composition D was found to contain 61.0% total dietary fiber, of which 96% was soluble dietary fiber. In contrast, Composition E contained 51.5% total dietary fiber, which consisted entirely of insoluble dietary fiber.

The chemical analysis of Compositions D and E were done in the manner described in Example 7, with results as shown in Tables 10 and 11.

TABLE 10

| | |
|---|---|
| Composition D | Composition E |

| Component | Mol % | % w/w | Mol % | % w/w |
|---|---|---|---|---|
| Carbohydrate | | | | |
| Arabinose | 22.9 | 16.4 | 10.5 | 5.3 |
| Xylose | 42.8 | 30.8 | 26.5 | 13.2 |
| Mannose | 6.6 | 5.7 | 1.9 | 1.1 |
| Galactose | 4.9 | 4.2 | 3.5 | 2.1 |
| Glucose | 2.3 | 2.0 | 43.9 | 26.4 |
| Rhamnose/Fucose | 0.1 | 0.1 | — | 0 |
| Uronic acid | 20.2 | 18.7 | 13.7 | 8.9 |
| Lignin | | 3.2 | | 11.8 |
| Fat | | 3.7 | | 8.1 |
| Protein | | 6.3 | | 13.8 |
| Ash | | 2.8 | | 2.3 |
| Moisture | | 6.1 | | 7.0 |

TABLE 11

| | Ferulic acid (% w/w) | | Phenolics (% w/w) | | ORAC value (μmol TE/g) | |
|---|---|---|---|---|---|---|
| Sample | Free | Total | Free | Total | Free | Total |
| Composition D | 0.001 | 2.4 | 0.02 | 3.5 | 5.6 | 237.5 |
| Composition E | 0.008 | 1.4 | 0.1 | 2.4 | 46.1 | 212.4 |

Comparison of Tables 10 and 11 with Tables 7 and 8 shows that soluble fiber extracted in this trial was subtly different than the earlier product. The hemicellulose component of Composition D contained a higher proportion of hydrophilic arabinose side-groups in relation to the xylose backbone than that of Composition C. Composition D also contained a smaller proportion of phenolic groups, with lower overall antioxidant activity, than Composition C. Together, these results suggest that the hemicellulose extracted at 145° C. for 3 minutes was more water soluble than that extracted at 120° C. for 1.5 hours, due to the presence of a higher proportion of arabinose side-chains free from associated phenolic groups.

Composition D was also found to contain somewhat more water-soluble pectin than Composition C.

In contrast, the monosaccharides in Composition E was found to be predominantly glucose, along with some arabinose, xylose and uronic acid. This suggests that Composition E is mainly comprised of insoluble cellulose, as well as hemicellulose and a small amount of pectin trapped within the cellulose matrix.

Despite the relatively low level of hemicellulose, Composition E was found to contain a significant amount of antioxidant phenolic compounds. Composition E was also especially rich in lignin, which is an amorphous, plastic-like phenolic polymer which fills the spaces in the matrix of cell wall polysaccharides. Lignins are known to have antioxidant activity (Barclay et al J. Wood Chem. Technol. 17 (1997) 73-90).

This example has served to show that continuous, high-temperature cooking can be a viable processing alternative to batch retorting in cans. This would be the most appropriate method for commercial practice.

This example has also shown that a second product could be produced as a by-product from soluble fiber extraction, comprised of predominantly insoluble dietary fiber with a high proportion of antioxidant-rich lignin. Such a product may find application as an antioxidant fruit fiber where solubility is not a necessary feature. For example, it may find applications in processed fruit pieces, snack bars and baked goods.

### Example 10

### Application in Juice

Composition C was added to commercial apple juice at 12 g/L, in the manner described in Comparison 1. The fiber imparted a pale golden-brown color to the juice and a slight “cloud”, but the fortified juice had essentially no apparent increase in viscosity and no indication of a gummy mouthfeel. Instead, the pineapple fiber conferred a velvety, smooth consistency which tended to mellow the flavour of the rather sharp apple juice.

The fortified juice was very stable, with no indication of settling after several weeks.

For comparison, similar juice preparations were prepared with three commercial dietary fiber preparations, all in apple juice at 12 g/L. This concentration was selected to provide a “good source of fiber”, equivalent to 3 g per 250 mL serve. The three commercial products were Glucagel (barley beta-glucan), Herbapekt SF02-LV (pectin with viscosity reduced 20-fold) and Herbapekt SF50-A-LV (apple pectin with viscosity reduced 50-fold).

It was found that all three commercial products produced a noticeable increase in the viscosity of the apple juice, resulting in an objectionable ‘slimy’ or ‘gummy’ mouthfeel. In contrast, the pineapple fiber had a much more pleasant mouthfeel, lacking any slimy characteristic.

Surmacka Szczesniak and Farkas [J. Food Sci. 27 (1962) 381-385] reported that gums that are very slimy in the mouth can be characterised as exhibiting near-Newtonian rheological behaviour, whereas gums that are highly shear-thinning are non-slimy. Accordingly, the viscosity of the four apple juice preparations was measured as a function of shear rate, using a Brookfield DVII+ viscometer with a SC4-18/13R small volume spindle. The resulting viscosity profiles are shown in FIG. 4.

These results show that all four fiber solutions were strongly shear-thinning at very low shear rates, while they all exhibited near-Newtonian behaviour at shear rates greater than about 10 sec⁻¹. This suggests that all four fibers have similar ‘slimy’ mouthfeel characteristics under practical conditions. However, at a concentration typical of what might be used in juice commercially, the pineapple fiber had the lowest viscosity. This characteristic is apparently responsible for the superior mouthfeel reported in taste tests.

We were interested to learn whether the antioxidant activity associated with the soluble fiber of this invention would make a significant difference to the total antioxidant capacity of commercial fruit juices, when added at levels appropriate for making a dietary fiber claim.

Accordingly, samples of commercial shelf-stable fruit juices were fortified with 3 g of antioxidant fiber (Composition C) per 250 mL serving. The samples were frozen and sent to Southern Cross University (Lismore, NSW, Australia) for analysis of both total ORAC value and ascorbic acid content (by HPLC).

Each of the commercial juices had been fortified with ascorbic acid, which contributed to the total antioxidant activity. In order to focus solely on the antioxidant effect of phenolic compounds, the ORAC values were corrected for the contribution due to ascorbic acid. Control samples of ascorbic acid in water were analysed, to establish the necessary correction factor. The corrected ORAC values for control and fortified juices are shown in Table 12.

TABLE 12

ORAC value (μmol TE/mL)

| | | Juice plus 3 g/250 mL |
|---|---|---|
| Juice type | Juice | antioxidant fiber |
| Apple | 5.03 | 9.85 |
| Cranberry | 8.47 | 9.94 |
| Pineapple | 4.26 | 15.65 |

Since Composition C contains a total antioxidant content of 464.6 μmol TE/g (Table 8), fortification with 3 g/250 mL serve would be expected to increase the ORAC value of each juice by 5.58 μmol TE/mL. The results shown in Table 12 are broadly consistent with this expectation, but show a significant degree of scatter, presumably due to experimental error in the ORAC assay.

This data suggests that fortification of both apple juice and pineapple juice with 3 g antioxidant fiber per 250 mL serve effectively doubles the antioxidant activity of each juice. The resulting antioxidant activity is greater than that of cranberry juice, which is promoted as a rich source of dietary antioxidants. Fortification of cranberry juice does not have such a significant effect, since it starts from a higher antioxidant baseline level.

In commercial applications, it is important that the fiber product remains stable for the duration of the expected shelf-life. This is a particular problem for inulin, a widely-used dietary fiber, which is known to hydrolyse to fructose under the conditions of temperature and pH encountered during pasteurization and storage of acidic juice products [Blecker et al J. Agric. Food Chem. 50 (2002) 1602-1607]. This can result in the dietary fiber content progressively falling and the sweetness progressively rising during extended shelf storage.

In order to evaluate the shelf stability of pineapple fiber, Composition C was formulated in apple juice at 12 g/L as described above. For comparison, a similar juice mixture with Beneo GR, a commercial short-chain inulin, was also prepared. An accelerated shelf-life trial was conducted, which involved heating the samples at 80° C. for up to 2 days. These test conditions are based on the observations that the phenolic compounds in apple juice are halved in concentration by storage at 25° C. for 9 months [Spanos et al J. Agric. Food Chem. 38 (1990) 1572-1579], and that a similar decrease can be achieved by heating at 80° C. for 2 days [Van der Sluis et al J. Agric. Food Chem. 53 (2005) 1073-1080]. Hence, 2 days of heating apple juice at 80° C. is equivalent to storage for 9 months at room temperature.

Samples were taken daily and analysed for dietary fiber content at BRI Research Pty Ltd (Sydney, NSW, Australia). Total dietary fiber content of samples containing Composition C was determined using AOAC Official Method 991.43. The concentration of inulin (fructans) was determined using AOAC Official Method 997.08, and the concentration of fructose was determined by HPLC. Samples were also analysed for ferulic acid content by HPLC determination at Southern Cross University (Lismore, NSW, Australia).

The fiber and inulin analysis results are shown in Table 13 (average of duplicate samples).

TABLE 13

| | |
|---|---|
| Composition C | Beneo GR |

| Time | Total dietary | Inulin % | |
|---|---|---|---|
| (days) | fiber % | (as Fructans) | Fructose % |
| 0 | 0.25 | 0.8 | 4.8 |
| 1 | 0.45 | 0.2 | 6.0 |
| 2 | 0.4 | <0.05 | 5.6 |

These results indicate that Composition C is stable in apple juice (pH 3.4) at 80° C. for 2 days. In contrast, the concentration of inulin fell by 75% over the first day and was below the detection limit by the second day.

The ferulic acid (free and total) content of the juice samples fortified with Composition C is shown in Table 14.

TABLE 14

Ferulic acid
(mg/mL)

| Day | Free | Total |
|---|---|---|
| 0 | tr. | 0.24 |
| 1 | tr. | 0.23 |
| 2 | tr. | 0.25 |

These results show that the ferulic acid component of Composition C remained covalently bound to the fiber throughout 2 days of heating at 80° C., with no detectable release of free ferulic acid into the juice. This is in contrast to the phenolic components of the apple juice base, which are known to be halved in concentration by this treatment.

This example demonstrates that the antioxidants associated with the soluble fiber of this invention are likely to remain stable for up to 9 months of storage at room temperature, and will also survive pasteurization intact.

The accelerated storage trial results in this example have shown that both the dietary fiber and the antioxidant component of the soluble fiber of this invention are highly stable in acidic juice beverages, particularly those that are pasteurized to ensure a long shelf-life. As such, this product offers distinct advantages over inulin, which breaks down rapidly under acidic conditions, and free phenolic antioxidants which also degrade in juice over time.

### Example 11

### Application in Milk

Composition C was dispersed into cold milk at 12 g/L using a kitchen blender and was stored under refrigeration. There was no sign of phase separation even after 7 days of storage. This preliminary result suggests that there is no practical phase incompatibility between pineapple fiber and milk. Hence, it may be expected that the soluble fiber of this invention could be used as an ingredient in a range of milk products, as a dietary fiber supplement or as a prebiotic ingredient.

### Example 12

### Application in Yoghurt

Composition C was formulated in natural-set yoghurt at a concentration equivalent to 1.5 g per 200 mL serve, i.e. 7.5 g/L, sufficient to provide “a source of fiber”.

The yoghurt was prepared by adding 30 g skim milk powder and 7.5 g Composition C to 1 L fresh whole milk. The milk was heated to 90° C. by microwaving. The milk was cooled slowly to 42° C., at which time one tablespoon of commercial aBc (Acidophilus, Bifidus, Casei) yoghurt (Jalna Natural Yoghourt) was mixed in. The mixture was incubated at 42° C. for 6 hours to set, at which time the yoghurt was refrigerated. A control yoghurt lacking Composition C was also prepared. The samples were stored under refrigeration for 7 days before evaluation.

The integrity of the physical structure of the two yoghurts was assessed by measuring syneresis according to Aryana [Int. J. Dairy Technol. 56 (2003) 219-222]. A 300 g sample of each yoghurt was placed on a fine nylon mesh screen placed on top of a funnel and allowed to drain for 2 hours at 22° C. The quantity of whey collected was used as an index of syneresis. The analysis was conducted in duplicate, with results as shown in Table 12.

TABLE 15

| Sample | Average volume of whey (mL) |
|---|---|
| Control yoghurt | 125 |
| Yoghurt with Composition B | 106 |

It was observed that syneresis was about 20% greater in the control yoghurt than in the yoghurt containing pineapple fiber. The reason for this difference is unclear, although it may be due to the water-binding capacity of the pineapple fiber or to the presence of the fiber preventing coalescence of casein particles.

In order to gain further insight into the relative differences between the two samples, samples of the two yoghurts were tasted by two untrained testers. It was reported that the mouthfeel texture of both yoghurts seemed equally smooth. However, the control yoghurt had a sharp, lactic acid taste, whereas the yoghurt containing pineapple fiber was reported to be much smoother, having noticeably less sharpness and lactic acid taste.

These results suggest that the soluble fiber of this invention could be advantageously used as a fiber supplement or prebiotic ingredient in natural-set yoghurts, thickened pumping yoghurts, drinking yoghurts and other acidified dairy beverages.

### Example 13

### Application in Baked Goods

As an example of the application of pineapple fiber in baked goods, Composition C was formulated into muffins. For the sake of comparison, four different batches of muffins were prepared, according to the recipes shown in Table 13. Batch 1 was a control, with no fiber added. Batch 2 was formulated to deliver 1.5 g of Composition C per muffin. Batch 3 was formulated to contain 5 blueberries per muffin. Batch 4 was formulated to contain 1.5 g of wheat bran per muffin.

TABLE 16

| Ingredient | Batch 1 | | Batch 2 | | Batch 3 | | Batch 4 | |
|---|---|---|---|---|---|---|---|---|
| Plain white flour | 90 | g | 90 | g | 90 | g | 90 | g |
| Sugar | 30 | g | 30 | g | 30 | g | 30 | g |
| Baking powder | 2.7 | g | 2.7 | g | 2.7 | g | 2.7 | g |
| Salt | 0.7 | g | 0.7 | g | 0.7 | g | 0.7 | g |
| Eggs | 1 | | 1 | | 1 | | 1 | |
| Butter | 25 | g | 25 | g | 25 | g | 25 | g |
| Milk | 88 | g | 88 | g | 88 | g | 88 | g |
| Vanilla essence | 3 | mL | 3 | mL | 3 | mL | 3 | mL |
| Composition C | | | 9 | g | | | | |
| Blueberries | | | | | 75.7 | g | | |
| Wheat bran | | | | | | | 9 | g |

In each case, the dry ingredients were sifted together and then whisked with the eggs, milk, melted butter and vanilla essence. For Batch 3, the blueberries were carefully folded into the wet mixture. The mixture was dispensed into a greased muffin pan and baked in a preheated fan-forced gas oven at 200° C. All four batches were baked for 17 minutes.

Each batch of 6 muffins was cooled, frozen overnight, then transferred into a freeze-dryer. The dried muffins were weighed, pulverized in a household food processor, then sent to Southern Cross University (Lismore, NSW, Australia) for antioxidant analysis. The results are shown in Table 17.

TABLE 17

| | Total dry weight | ORAC | ORAC/muffin |
|---|---|---|---|
| Batch | (g) | (μmol TE/g) | (μmol TE) |
| 1 | 160 | 10.6 | 283 |
| 2 | 169 | 13.8 | 389 |
| 3 | 170 | 9.9 | 280 |
| 4 | 168 | 13.8 | 386 |

The results show that the control muffins (Batch 1) have a significant level of antioxidant activity. Some of this would be due to phenolic compounds present in the refined wheat flour, but a large proportion would also be due to Maillard reaction products formed during the cooking process [Yilmaz & Toledo Food Chem. 93 (2005) 273-278].

The muffins containing Composition C (Batch 2) were found to have the same antioxidant activity as those containing an equivalent amount of wheat bran (Batch 4). Interestingly, the muffins containing blueberries (Batch 3) had the same antioxidant activity as the control, suggesting that the blueberry anthocyanins had been destroyed during cooking. Such a high degree of thermal instability has also been noted for purple wheat bran anthocyanins baked into muffins (Li et al Food Chem. 104 (2007) 1080-1086].

The thermal stability of the covalently-bound ferulic acid in the soluble dietary fiber of this invention offers a real advantage over unstable berry anthocyanins in baked goods. In baked goods, the antioxidant activity of the soluble dietary fiber of this invention is the same as an equivalent amount of wheat bran. This suggests that addition of the antioxidant-rich soluble fiber of this invention to baked goods would confer some of the health benefits associated with wheat bran.

These results show that the soluble dietary fiber of this invention can be used in baked goods to deliver heat-stable antioxidant capacity from a fruit source.

### Example 14

### Emulsifying Properties

Hemicelluloses extracted from wheat bran (Schooneveld-Bergmans et al J. Cereal Sci. 29 (1999) 49-61) and corn fiber (Yadav et al Food Hydrocoll. 21 (2007) 1022-1030; Carvajal-Millan et al Carbohydr. Polym. 69 (2007) 280-285) have been shown to have emulsifying properties, but there have been no such results reported for pineapple hemicellulose.

In order to demonstrate the emulsifying properties of the soluble dietary fiber of this invention, we prepared a series of emulsions of fish oil in water. As a control, we used a commercial corn fiber product, Promitor Soluble Corn Fiber from Tate & Lyle (London, UK) which has no associated antioxidant activity.

The fish oil was a refined tuna oil (HiDHA® 25N Food) from NU-MEGA INGREDIENTS Pty Ltd (Altona North, Victoria, Australia). The tuna oil was emulsified with a 40% w/w solution of either Composition C or Promitor Soluble Corn Fiber, plus additional water, to make six different emulsion recipes, as shown in Table 18.

TABLE 18

Proportion of mixture, % w/w

| | Pineapple fiber | | | Corn fiber | | |
|---|---|---|---|---|---|---|
| Ingredient | #1 | #2 | #3 | #4 | #5 | #6 |
| Fiber | 14 | 7 | 3.5 | 14 | 7 | 3.5 |
| Tuna oil | 30 | 30 | 30 | 30 | 30 | 30 |
| Water | 56 | 63 | 66.5 | 56 | 63 | 66.5 |

The emulsions were prepared in 50 g batches, using a Ystral T1500 high-shear mixer with a YS3910F head, operated at setting 8 for 60 seconds. A preliminary evaluation of the stability of the emulsions was undertaken by initial microscopic evaluation of the droplets, followed by standing overnight at room temperature.

Emulsion 1 was microscopically observed to be comprised of a uniform suspension of very small droplets. After standing overnight, there was no sign of oil separation. Emulsion 2 was observed to be a mixture of mostly small droplets and a small percentage of large droplets. No oil separation was evident after standing overnight. Emulsion 3 was observed to comprise predominantly large droplets. A thin layer of tuna oil separated from the emulsion after standing overnight. Emulsions 4, 5 and 6 were all observed to be comprised of large droplets, which were not stable even during observation. A thick layer of tuna oil separated from oil three emulsions during overnight standing.

This preliminary investigation suggested that an emulsion of 14% w/w Composition C and 30% w/w tuna oil was likely to be stable. An emulsion containing 7% Composition C may also be stable in some applications, but may possibly be less stable over time. An emulsion containing 3.5% Composition C was clearly unstable. None of the emulsions containing Promitor Soluble Corn Fiber were observed to be stable.

This preliminary investigation was followed by a more formal evaluation of the stability of fish oil emulsions prepared with the soluble dietary fiber of this invention. In this case, emulsions containing Promitor Soluble Corn Fiber were supplemented with the surfactant sodium dodecyl sulphate (SDS) to ensure emulsion stability. Potassium sorbate was added as a preservative. Emulsions of each fiber were prepared either with or without disodium EDTA. The recipes for the emulsions are shown in Table 19.

TABLE 19

| Proportion, % w/w | |
|---|---|
| Pineapple | Corn |
| fiber | fiber |

| Ingredient | # 1 | # 2 | # 3 | # 4 |
|---|---|---|---|---|
| Tuna oil | 30.0 | 30.0 | 30.0 | 30.0 |
| Fiber | 14.0 | 14.0 | 14.0 | 14.0 |
| SDS | | | 0.5 | 0.5 |
| Potassium sorbate | 0.05 | 0.05 | 0.05 | 0.05 |
| Disodium EDTA | | 0.03 | | 0.03 |
| Water | 69.95 | 69.92 | 69.45 | 69.42 |

The emulsions were prepared with high-shear blending as described above. For evaluation of emulsion stability, the emulsions were stored at 40° C. for 4 weeks. The size distribution of the droplets in the emulsions was measured at the Department of Chemical and Biomolecular Engineering, University of Melbourne (Parkville, Victoria, Australia), using a Malvern Series 4700 spectrometer (Malvern Instruments Ltd, Malvern UK) with a 488 nm Argon Ion laser operating at 10 mW.

The mean droplet size in each of the emulsions both initially and after 4 weeks at 40° C. are shown in Table 20.

TABLE 20

Mean droplet
size, μm

| Emulsion | T = 0 | T = 4 weeks |
|---|---|---|
| # 1 | 1.06 | 1.32 |
| # 2 | 1.08 | 2.16 |
| # 3 | 0.69 | 0.73 |
| # 4 | 0.38 | 0.62 |

The droplet sizes in all of the emulsions increased slightly upon storage for 4 weeks at 40° C., but all remained stable with no sign of a free oil phase. Therefore, the pineapple fiber was found to act as an effective emulsifier for tuna oil in water.

This example demonstrates that the soluble dietary fiber of this invention can act as an effective emulsifier for oil-in-water emulsion systems, suggesting that it could find applications such as mayonnaise, salad dressings, beverages and as an encapsulating agent.

### Example 15

### Prevention of Lipid Oxidation

Fish oils are rich in docosahexaenoic acid (DHA) and eicosapentaenoic acid (EPA), which are susceptible to oxidation because of the multiple double bonds in their carbon chains. Lipid oxidation in oil-in-water emulsions is highly dependent on the interfacial characteristics of the lipid droplets, since this is the location where transition metals and lipid hydroperoxides interact to form damaging free radicals. Lipophilic antioxidants are more effective than hydrophilic antioxidants in oil-in water emulsions, because they concentrate at the interface to provide protection at the site of free radical formation (Frankel et al J. Agric. Food Chem. 42 (1994) 1054-1059).

Ferulic acid is known to be relatively lipohilic (Jacobsen et al J. Agric. Food Chem. 47 (1997) 3601-3610). Therefore, we were interested to see whether the antioxidant ferulic acid groups in the soluble dietary fiber of the present invention would be able to prevent oxidation of a fish oil emulsion.

Accordingly, replicate samples were prepared of the four tuna oil emulsions described in Table 19. The emulsions were prepared in the manner described in Example 14 and were subjected to an accelerated storage trial. This consisted in storing the emulsions in large, wide jars at 40° C. in the dark. The jars were usually kept closed to prevent evaporation of water, but were opened every 7 days to allow fresh air into the headspace. Over a one month period, jars were taken out of the incubator and stored frozen at −20° C. pending analysis.

As a parallel experiment, bottles of commercial apple juice were fortified with each of the four emulsions at a dilution ratio of 1:10, giving a tuna oil concentration of 3 g/L. The juice samples were stored in closed bottles, at 40° C. in the dark. Five replicates of each were prepared. Each week, one bottle was opened and assessed subjectively for the development of fishy odours.

As an additional test to simulate real-world conditions, bottles of each of the fortified juice samples were stored in a refrigerator for 8 weeks before sampling.

The presence of oxidation products in the frozen emulsion samples was quantified by Food Science Australia (Werribee, Victoria, Australia) using the methods of Richards et al JAOCS 82 (2005) 869-874.

GC-headspace analysis of propanal is reported to be an excellent method for following the oxidation of n-3 fatty acids such as DHA (Boyd et al JAOCS 69 (1992) 325-330). Table 21 shows the relative abundance of propanal in each of the emulsions taken from accelerated storage.

TABLE 21

Propanal concentration
(abundance of ion 56 m/z)

| | Pineapple | Pineapple | Corn | Corn |
|---|---|---|---|---|
| Time, | Fiber − | Fiber + | Fiber − | Fiber + |
| weeks | EDTA | EDTA | EDTA | EDTA |
| 0 | 10,848 | 2,565 | 8,163 | 775 |
| 1 | 4,052 | 1,423 | 17,858 | 1,004 |
| 2 | 42,792 | 24,411 | 171,808 | 106,143 |
| 3 | 67,343 | 63,514 | 282,099 | 473,693 |
| 4 | 88,575 | 90,579 | 446,205 | 437,047 |

It was found that the concentration of propanal was much lower in the emulsions containing pineapple fiber, indicating that the antioxidant groups associated with the soluble fiber were able to effectively protect the tuna oil from oxidation. In contrast, the corn fiber product having no antioxidant activity provided no such protection.

The human sense of smell is an extremely sensitive tool for detecting fishy or rancid odors in oils. It is this that will dictate whether an oil-containing product will be acceptable to the consumer. The results of the subjective analysis of the juice samples subjected to accelerated storage are shown in Table 22. The results are rated in relative terms with (−) denoting an absence of fishy odors, and (+++) denoting a strong fishy smell.

TABLE 22

| | Pineapple | Pineapple | Corn | Corn |
|---|---|---|---|---|
| Time, | Fiber − | Fiber + | Fiber − | Fiber + |
| weeks | EDTA | EDTA | EDTA | EDTA |
| 0 | − | − | − | − |
| 1 | − | − | + | − |
| 2 | + | − | ++ | + |
| 3 | + | − | +++ | ++ |
| 4 | + | − | +++ | ++ |

Apple juice contains ferrous ions, which promote free radical formation and thus oxidation of oils. EDTA was added to the samples to complex with ferrous ions and inhibit oil oxidation.

In the absence of EDTA, the pineapple fiber did not afford complete protection against the development of fishy smells. However, the combination of pineapple fiber plus EDTA was able to prevent the development of fishy odors during the 4 week accelerated storage trial.

In contrast, the corn fiber with no antioxidant activity was unable to prevent the development of fishy odors, even with EDTA present.

This example shows that the soluble dietary fiber of the present invention can provide a substantial benefit in oil-in-water emulsion systems, by preventing oil oxidation and the development of off-odors. This suggests that the soluble dietary fiber of the present invention can be of commercial benefit in the formulation of sensitive oils such as fish oils and essential oils, in applications such as beverages and flavor formulations.

1. A method of preparing an antioxidant dietary fiber comprising:
heating pineapple pulp in an autoclave to a temperature in the range from 105° C. to 150° C., for a period of at least 30 seconds;
providing a pH of the autoclaved pineapple pulp in the range of from 3.2 to 6.5;
subjecting the heated material to mechanical or ultrasonic treatment to facilitate removal of soluble material from the solid material;
separation of the solubilised fiber from insoluble material;
at least partly removing free sugars; and
concentrating the soluble antioxidant fiber.

2. A method according to claim 1 wherein the pineapple pulp is heated to within the range of from 90° C. to 150° C. for a period of at least ten minutes including said heating pineapple pulp to a temperature in the range from 105° C. to 150° C., for a period of at least 30 seconds.

3. A method according to claim 1 wherein the resulting dietary fiber has an antioxidant activity of greater than 50 micromoles Trolox equivalent per gram (ORAC) due to the presence of covalently bound ferulic acid and p-coumaric acid.

4. A method according to claim 1 wherein the pineapple pulp is a food-grade by-product of a commercial juicing operation, such as discarded skin, core or clarifier centrifuge sludge.

5. A method according to claim 1 wherein the method further comprises washing the pineapple pulp prior to extraction whereby the extent of color formation is reduced.

6. A method according to claim 1 wherein the pineapple pulp comprises pulp particles of size in the range of from 0.5 mm to 50 mm.

7. A method according to claim 1 wherein the pineapple pulp is admixed with a quantity of water to provide a weight ratio of water to pulp of from 0.5:1 to 5:1.

8. A method according to claim 1 comprising the addition of alkali to the pineapple pulp prior to heating the pineapple pulp.

9. A method according to claim 1 wherein the heating of the pulp is provided by steam injection, indirect steam heating or microwaving of the pulp.

10. A method according to claim 1 wherein the pulp is subject to ultrasonic treatment during or after heating to facilitate removal of the soluble material from the solids.

11. A method according to claim 10 wherein the ultrasonic treatment is conducted by treating the cooked pulp slurry in a continuous manner in a flow-through ultrasonic chamber.

12. A method according to claim 1 wherein the separation of the soluble and insoluble materials is carried out by a method selected from the group consisting of pressing, filtration, and gravity settling.

13. A method according to claim 1 wherein the water soluble fiber is concentrated by a process selected from the group consisting microfiltration, ultrafiltration, nanofiltration and reverse osmosis.

14. A method according to claim 1 wherein the water soluble fiber is concentrated by a process of microfiltration using a membrane having a pore size of between 0.1 micron and 1 micron.

15. A method according to claim 13 wherein concentration of the solubilised fiber comprises ultrafiltration using an ultrafiltration membrane with a molecular weight cut-off in the range of between 1,000 and 100,000.

16. A method according to claim 1 wherein the at least partial removal of sugars from the solubilised fiber comprises diafiltration conducted to the extent that the ash level of the pineapple fiber product is between 1% and 5% on a dry matter basis.

17. A method according to claim 1 wherein the process of concentrating the water soluble antioxidant fiber comprises drying to form a particulate solid.

18. A method according to claim 17 wherein the water soluble antioxidant fiber is dried using a method selected from the group consisting of spray drying, freeze drying and drum drying.

19. A method according to claim 1 further comprising addition of stabilisers to prevent browning of the fiber during processing.

20. A method according to claim 19 comprising addition of stabilisers selected from the group consisting of sulphur dioxide in the form of sulphur (iv); and oxyanines such as HSO₃⁻, SO₃²⁻.

21. A method according to claim 1 further comprising addition of sodium metabisulphite stabiliser to the pineapple pulp prior to heating the pineapple pulp in an autoclave.

22. A method according to claim 1 wherein the amount of sodium metabisulphite is in the range of from 10 to 1,000 ppm and most preferably in the range of from 100 to 300 ppm.

23. A method according to claim 1 further comprising addition of ascorbic acid stabiliser in an amount in the range of from 10 to 2,000 ppm.

24. A method according to claim 1 wherein the solubilised fiber is treated with activated carbon to remove free phenolic compounds.

25. A method according to claim 1 further comprising isolating the insoluble fiber from the insoluble material.

26. A soluble antioxidant fiber obtained from pineapple.

27. A soluble antioxidant fiber according to claim 26 wherein the dietary fiber has an antioxidant activity greater than 50 micromoles Trolox equivalent per gram (ORAC) due to the presence of covalently bound ferulic acid and p-coumaric acid, preferably in the range of 100 to 1000 micromoles Trolox equivalent per gram, and most preferably in the range of 200 to 800 micromoles Trolox equivalent per gram.

28. A soluble antioxidant fiber according to claim 26 wherein the soluble fiber product comprises 75% to 99% carbohydrate, 0.5% to 5% lignin, 0.5% to 5% fat, 1% to 10% protein and 1% to 5% ash, on a dry weight basis, and most preferably 80% to 95% carbohydrate, 1.5% to 3.5% lignin, 1% to 4% fat, 2% to 7% protein and 1.5% to 3% ash.

29. A soluble antioxidant fiber according to claim 26 wherein the carbohydrate component is comprised of 60% to 90% total dietary fiber (as measured by AOAC Official Method 991.43) and 10% to 40% sugars and oligosaccharides.

30. A soluble antioxidant fiber according to claim 26 wherein the fiber is present in a composition comprising a total dietary fiber comprised of 90% to 100% soluble dietary fiber and 0% to 10% insoluble dietary fiber (as measured by AOAC Official Method 991.43).

31. A soluble antioxidant fiber according to claim 26 wherein the carbohydrate component of the pineapple fiber product is comprised of 40-80% xylose, 5-25% arabinose, 2-15% galactose, 0.1-15% glucose, 0.1-10% mannose, 0-2% rhamnose/fucose and 5-25% uronic acid, on a mole-percentage basis.

32. A food composition comprising a soluble dietary fiber according to claim 26.

33. A food composition comprising a soluble dietary fiber according to claim 32 wherein the food composition is selected from the group consisting of drinks, dairy products, soy and grain milks, soups, baked goods, breakfast cereals and snack bars, meat products, emulsified edible oils, encapsulated edible oils, instant drinks, instant desserts and soup mixes.

34. A food composition comprising a soluble dietary fiber according to claim 32 in the form of a fruit and vegetable juices, milk drinks, soy milk, rice milk, drinking yoghurt and other acidified dairy drinks.

35. A food composition according to claim 32 comprising a soluble dietary fiber wherein the soluble dietary fiber is added to a beverage in an amount of at least 0.1 gram per litre.

36. A food composition according to claim 32 comprising the soluble dietary fiber as an emulsifying agent for edible oils.

37. A food composition according to claim 32 comprising the soluble dietary fiber as an encapsulating agent for edible oils.

38. A food composition according to claim 36 is selected from the group consisting of fish oils, microalgal oils, single-cell omega-3 fatty acids, seed oils, nut oils, essential oils, flavors and aromas.

39. A food supplement comprising a soluble dietary fiber according to claim 26.

40. A cosmetic composition comprising a soluble dietary fiber according to claim 26.

41. A pharmaceutical composition comprising a soluble dietary fiber according to claim 26.
